(19) United States
(12) Patent Application Publication
(10) Pub. No.: US 2026/0284660 A1
(43) Pub. Date: Sep. 24, 2026

(54) LIQUID DISPENSING ROBOT, LIQUID DISPENSING ROBOTIC SYSTEM AND METHOD OF LIQUID DISPENSING

(71) Applicant: Nanyang Technological University, Singapore (SG)

(72) Inventors: Guo Zhan Lum, Singapore (SG); Zilin Yang, Singapore (SG); Changyu Xu, Singapore (SG)

(21) Appl. No.: 19/555,733

(22) Filed: Mar. 3, 2026

(30) Foreign Application Priority Data
Mar. 11, 2025 (SG) 10202500615R

(51) Int. Cl. B01L 3/00; B25J 7/00; B25J 9/12; B25J 11/00

(52) U.S. Cl. CPC B01L 3/50273; B25J 7/00; B25J 9/12; B25J 11/009; B01L 2400/043

(57) ABSTRACT

A liquid dispensing robot. The liquid dispensing robot includes a plurality of dispensing modules, each including: an housing defining an inner chamber and an opening, the opening allowing fluid communication between the inner chamber and an environment. Each of the plurality of dispensing modules further includes a dispensing member disposed in the inner chamber adjacent to the opening and an actuating member disposed in the inner chamber, where the actuating member defines an actuation frequency band. Responsive to a control magnetic field with a dispensing frequency within the actuation frequency band, the actuating member deforms to periodically displace the dispensing member relative to the opening to vary a local fluid pressure adjacent to the opening, where each of the actuation frequency band is non-overlapping with one another, and where each of the actuating member of the plurality of dispensing modules is selectively actuatable by the control magnetic field.

### TECHNICAL FIELD

This application relates to the field of liquid dispensing, and more particularly to a liquid dispensing robot, a liquid dispensing robotic system and a method of liquid dispensing for drug delivery.

### BACKGROUND

Targeted drug delivery is a class of medical treatments that can significantly enhance the concentration of drugs at specific locations in the human body. Miniature robots provide a possible avenue for drug delivery. However, it is challenging to effectively control such miniature robots for complex drug delivery procedures.

### SUMMARY

According to an aspect, disclosed herein is a liquid dispensing robot. The liquid dispensing robot comprises a plurality of dispensing modules, each of the plurality of dispensing modules comprising: a housing defining an inner chamber and an opening, the opening allowing fluid communication between the inner chamber and an environment; an actuating member moveably coupled to the housing, the actuating member disposed in the inner chamber defining an actuation frequency band; and a dispensing member movably coupled to the housing, the dispensing member disposed in the inner chamber adjacent to the opening, wherein responsive to a control magnetic field with a dispensing frequency within the actuation frequency band, the actuating member deforms to periodically displace the dispensing member relative to the opening to vary a local fluid pressure adjacent to the opening, wherein each of the actuation frequency band is non-overlapping with one another, wherein each of the actuating member of the plurality of dispensing modules is selectively actuatable by the control magnetic field.

According to another aspect, disclosed herein a liquid dispensing robotic system. The liquid dispensing robotic system comprises an electromagnetic coil system defining a workspace; and the liquid dispensing robot as described above disposed in the workspace, the electromagnetic coil system being configured to provide the control magnetic field.

According to yet another aspect, disclosed herein a method of liquid dispensing. The method comprising: providing a control magnetic field with a first dispensing frequency to the liquid dispensing robot as described above; and providing the control magnetic field with a second dispensing frequency to the liquid dispensing robot, wherein the first dispensing frequency is within a first actuation frequency band and the second dispensing frequency is within a second actuation frequency band, wherein the first actuation frequency band is non-overlapping with the second actuation frequency band.

### BRIEF DESCRIPTION OF THE DRAWINGS

Various embodiments of the present disclosure are described below with reference to the following drawings:

FIG. 1 is a perspective view of a liquid dispensing robotic system according to various embodiments;

FIG. 2 is a perspective view of a liquid dispensing robot according to various embodiments;

FIG. 3 is an exploded view of the liquid dispensing robot of FIG. 2;

FIG. 4 is a partial view of a dispensing module according to various embodiments;

FIG. 5 is a sectional view of a dispensing module according to various embodiments;

FIGS. 6A to 6C show a dispensing process of the dispensing module of FIG. 5;

FIG. 7 is a sectional view of a dispensing module according to various embodiments;

FIG. 8 is a sectional view of a dispensing module according to various embodiments;

FIG. 9 is a sectional view of the dispensing module of FIG. 8 under a control magnetic field with a quasistatic frequency according to various embodiments;

FIGS. 10A to 10C show different configurations of a liquid dispensing robot according to various embodiments;

FIG. 11 is a flowchart of a method of liquid dispensing according to various embodiments;

FIG. 12A shows a miniature soft robot of an exemplary embodiment configured to carry four distinct drugs, and being able to reprogram its drug-dispensing sequence and dosage. (i) An auxiliary view of soft robot, which shows that it has four drug modules and a pair of soft tentacles. Each drug module carries a distinct drug, i.e., the drugs in modules I, II, III and IV. (ii) The side view of the soft robot where the auxiliary magnet is between the drug modules and the soft tentacles, and its magnetic moment is parallel to \vec{m}. The magnetization profile of the soft tentacles is shown to be symmetrical. By having this magnetization profile, the soft tentacles can deform into an inverted “U”-shaped configuration and the soft robot's \vec{m} is always parallel to the applied \vec{B}. Also shown is the local coordinate frame of the soft robot here. The semi-circle outline is used as a reference for the soft robot's orientation;

FIG. 12B shows a front view of a drug module. (i) Each drug module has a pair of identical magnetic vibrating beams and valves with one opening. (ii) When the magnetic vibrating beams are subjected to a designated, alternating \vec{B} of selected f and Bₐₘₚ, they will vibrate and strike the valve in the module to eject the drugs. The semi-circle outline corresponds to that of FIG. 12A;

FIG. 12C shows the decoupling of the proposed soft robot between the locomotion and drug-dispensing mechanism, and it can also dispense its drugs selectively. (i) The soft robot first rolls from the center of the substrate to the first quadrant and dispenses drug I. (ii-iv) The soft robot then rolls to the remaining quadrants and dispenses a corresponding drug. The bottom legend indicates the corresponding arrow representations in the figure, scale bars: 1 mm;

FIG. 13A shows the proposed soft robot's six-DOF locomotion, the soft robot is commanded to rotate about its (i) x_{{L}}-, (ii) y_{{L}}- and (iii) z_{{L}}-axes;

FIG. 13B shows the soft robot translating along its (i) x_{{L}}-, (ii) y_{{L}}- and (iii) z_{{L}}-axes;

FIG. 13C shows the soft robot rolling along its (i-ii) length and (iii-iv) width. For both modes of rolling, the soft robot can be steered to follow an “X”-shaped path;

FIG. 13D shows the soft robot rolling along its length and width, and negotiating across barriers with strict shape constraints. A curved arrow is used to represent the instance in which the soft robot changes from rolling along its width to rolling along its length;

FIGS. 13E to 13G show the soft robot executing a two-anchor crawling gait to steer across an “L”-shaped path, ascend an inclined slope of 15° and negotiate across a barrier with strict shape constraints, respectively. The dotted lines represent the soft robot's trajectory, scale bars: 2 mm;

FIG. 14A shows the characterization of the soft robot's drug-dispensing performance with drugs I-IV. The drug modules of the soft robot were actuated by four sets of alternating \vec{B}. The experiments indicate that the drugs in each module will only be ejected if and only if their respective vibrating beams were subjected to a designated, alternating \vec{B} of selected f and Bₐₘₚ. Otherwise, the drugs remained in the modules;

FIG. 14B shows the drug concentration in the modules varying with respect to time after dispensing each drug module for 8 hours;

FIG. 14C shows the volume of drugs dispensed by drug modules I-IV with respect to time determined using computer vision, for the experiments in FIG. 14B. The corresponding data are shown in (i)-(iv), respectively. Each experiment was evaluated with three trials. The solid lines represent the mean volume of drugs dispensed by the modules, while the shaded regions represent their corresponding standard deviation. The data indicated that all the modules can dispense 0.282-0.586 μL of drugs within 2 hours and their mean dispensing rates were 0.144-0.231 μL/hour across 8 hours, scale bar: 2 mm;

FIG. 15A shows the characterization of the soft robot's drug-dispensing performance in a buffer solution and with fluorescent dye as the simulated drug for all the drug modules. The drug modules were actuated by four sets of alternating \vec{B} and each module only ejected its drugs if and only if its respective vibrating beams were subjected to a designated, alternating \vec{B} of selected f and Bₐₘₚ. Otherwise, the drugs remained in the modules, scale bar: 2 mm;

FIG. 15B shows the drug modules dispensing the respective drug over 8 hours. At an hourly interval, a fluorescence microscope recorded images of the drug module for determining the concentration of the drugs in the module at that time. Three examples of such fluorescent images are shown in FIG. 15B: the fluorescence images of drug module I at the 0-, 4- and 8-hour intervals of the experiment, scale bar: 200 μm;

FIG. 15C shows the volume of drugs dispensed by drug modules I-IV over 8 hours, respectively. All the experiments were evaluated with three trials. The centroid of the circles and their error bars represent the mean and standard deviation of these data points, respectively. The data indicated that all the modules can dispense 0.254-0.414 μL of drugs within 2 hours and their mean dispensing rates were 0.0992-0.115 μL/hour across 8 hours;

FIGS. 16A to 16J show the reprogramming of the soft robot's drug-dispensing sequence and dosage at three regions of the biological phantom. FIG. 16A shows the initial position of the soft robot as well as the locations of each region and their respective desired drug-dispensing sequence and dosage. Drugs I-IV were used in this experiment. FIG. 16B shows that by using two-anchor crawling gait, the soft robot was able to reach region I and dispense 0.0240 μL of drug I. (FIGS. 16C to 16D) Subsequently, the soft robot continued to dispense 0.116 μL of drug II and 0.0351 μL of drug IV at region I. FIGS. 16E to 16G show the soft robot rolling to region II and starts dispensing 0.0702 μL of drug IV, 0.0514 μL of drug III and 0.0240 μL of drug I sequentially. (FIGS. 16H to 16I) Once the desired drugs at region II were dispensed, the soft robot moved to region III and dispenses 0.0385 μL of drug II and 0.0480 μL of drug I sequentially. FIG. 16J shows the soft robot exiting the phantom by backtracking to the entry point, scale bar: 2 mm;

FIGS. 17A and 17B show the soft robot's deformation when \vec{B} of varying magnitudes were applied along the z_{{L}}-axis. FIG. 17A shows the tentacles' deformation when B_{z,{L}} was increased from 5 to 25 mT (i-v). Simulated results are represented by the white lines, which were superimposed onto the corresponding sub-figures. FIG. 17B shows the deformation of the vibrating beams in drug modules I-IV when B_{z,{L}} was varied from 1 to 25 mT. Simulated results are represented by the white lines, which were superimposed on the sub-figures. A characteristic of such deformation is that the vibrating beams bent away from the valve when \vec{B} was applied along the soft robot's \vec{m}, scale bars: 1 mm;

FIG. 18 shows the characterization on the leakage from the drug modules when they were not actuated in a buffer solution across an eight-hour period. All the drug modules were fully loaded with fluorescent dye. Each data point was evaluated with three trials, and the centroid and error bar of these data points represented their corresponding mean and standard deviations;

FIGS. 19A and 19B show the magnetization and fabrication process for the soft robot's tentacles and vibrating beams. FIG. 19A shows the steps of fabrication and magnetization the soft tentacles: (i) They were first molded and (ii) subsequently folded in half and magnetized by a uniform magnetic field of 1.1 T. (iii) Once the tentacles were allowed to recover to their original shape, the tentacles possess the desired magnetization profile, indicated by the collection of magnetic dipoles (arrows). FIG. 19B shows the steps of fabrication and magnetization the vibrating beams in a drug module: (i) the molded components were magnetized by a uniform magnetic field of 1.1 T as shown in (ii). (iii) The magnets were then bonded adhesively to the free ends of the beams, the magnetic moment of the magnets represented by the arrow;

FIG. 20 shows a customized electromagnetic coil system and its workspace, scale bar: 2 cm;

FIGS. 21A and 21B show the experimental setup and required calibration for evaluating the drug dispensing rates of our soft robot. FIG. 21A shows a camera being placed above the drug modules to read the gray scale levels of their encased drugs. FIG. 21B shows the relationship between the gray scale readings and the concentrations of the drugs. Each data point was evaluated with five trials. The centroid and error bars of the data points represented their corresponding mean and standard deviation;

FIG. 22 shows drugs I-III being carried by drug module IV, and with the drug-dispensing rate evaluated over a period of 8 hours;

FIGS. 23A and 23B show the experimental setup to calibrate the relationship between the drugs' gray scale reading and their respective concentration. FIG. 23A shows the experimental setup with a camera placed on top of the soft robot to read the gray scale levels of the drugs. FIG. 23B shows the relationship between the gray scale readings and the concentrations of the drugs. Based on the data points, a best fit cubic polynomial curve was fitted to describe such a relationship. Each data point was evaluated with five trials. The centroid and error bars of these data points represent their corresponding mean and standard deviations;

FIG. 24A shows the set-up of a fluorescence microscope with an excitation filter configured to allow electromagnetic waves of 465-495 nm wavelength from the source light to pass through and reach the drug module. When the fluorescence dye emitted electromagnetic waves of 480-650 nm wavelength, the emission filter would only allow electromagnetic waves of 515 nm wavelength to pass through and reach the fluorescent microscope;

FIG. 24B shows the experimental data that established the relationship between the emitted light intensity from the fluorescent dye and its concentration. Each data point was evaluated with three trials. The centroid and error bars of these data points represented their corresponding mean and standard deviations. The data was fitted with a best-fit quadratic function;

FIGS. 24C to 24E illustrate the fluorescence images of drug modules II, III and IV at the 0-, 4- and 8-hour intervals when they were dispensing the fluorescent dye across eight hours, respectively. The light intensity regions that overlapped with the robot's structure in the module were filtered off to obtain more accurate measurements for the drug-dispensing rate characterizations, scale bar: 200 μm; and

FIGS. 25 to 38 show various exemplary control magnetic signals for controlling the robot according to various embodiments.

### DETAILED DESCRIPTION

The following detailed description is made with reference to the accompanying drawings, showing details and embodiments of the present disclosure for the purposes of illustration. Features that are described in the context of an embodiment may correspondingly be applicable to the same or similar features in the other embodiments, even if not explicitly described in these other embodiments. Additions and/or combinations and/or alternatives as described for a feature in the context of an embodiment may correspondingly be applicable to the same or similar feature in the other embodiments.

In the context of various embodiments, the articles “a”, “an” and “the” as used with regard to a feature or element include a reference to one or more of the features or elements.

In the context of various embodiments, the term “about” or “approximately” as applied to a numeric value encompasses the exact value and a reasonable variance as generally understood in the relevant technical field, e.g., within 10% of the specified value.

As used herein, the term “and/or” includes any and all combinations of one or more of the associated listed items.

The term “quasistatic magnetic field” or “locomotion frequency” may generally correspond to a static or a slow changing magnetic field. An exemplary quasistatic magnetic field may be a static (non-alternating) magnetic field. In another example, the quasistatic magnetic field may be an alternating magnetic field with a frequency of 8 Hz or lower. In yet another example, the quasistatic magnetic field may have a frequency of 2 Hz or lower. The term “quasistatic magnetic field” may also be used interchangeably with the term “static magnetic field”.

The term “liquid-based drug” may generally refer to a liquid medicinal product, a solid medicinal product dissolved in a liquid base, a solid particle medicinal product mixed in a liquid base, a mixture of a solid particle medicinal product and a liquid medicinal product, a mixture of multiple liquid medicinal products, etc.

The present disclosure proposes a magnetically actuatable liquid dispensing robotic system and a liquid dispensing robot. In various embodiments, the liquid dispensing robotic system and the liquid dispensing robot may be used for liquid-based drug delivery. In various embodiments, the liquid-based drug delivery may be an in-vivo drug delivery procedure. The liquid dispensing robot may be loaded with multiple types of liquid-based drugs. The liquid dispensing robot may selectively or controllably dispense each of the liquid-based drugs based on a drug delivery plan/procedure.

In various embodiments, the liquid dispensing robotic system may include an electromagnetic coil system for controlling the liquid dispensing robot via a control magnetic field. For in-vivo drug delivery, the control magnetic field may have a magnetic field strength in a range of 2 to 25 millitesla (mT). In other embodiments, the control magnetic field may have a magnetic field strength in a broader range of 1 to 50 millitesla (mT).

In various embodiments, the liquid dispensing robot may include a plurality of dispensing modules each holding a liquid-based drug or a combination of drugs. The plurality of dispensing modules may be magnetically activated. Dispensing the liquid-based drug from each of the plurality of dispensing modules may be independently and selectively controlled using a control magnetic field. In various embodiments, dispensing the liquid-based drug from each of the plurality of dispensing modules may be activated by the control magnetic field with one or more dispensing frequencies. As each of the plurality of dispensing modules may be activated or controlled with a different and non-overlapping dispensing frequency, controlling the dispensing frequency or dispensing frequencies allows a controlled dispensing of one or more drugs from the plurality of dispensing modules.

Each of the liquid-based drugs in the plurality of dispensing modules may thus be dispensed at different time points. As examples, the liquid-based drugs may be dispensed sequentially, periodically, concurrently, partially concurrently or on demand. This allows fine-tuning of drug delivery such that a drug delivery plan may be optimized based on the timing of different drug delivery. In some examples, this enables a patient response to be observed prior to delivering a subsequent or further drug or drug dosage.

In various embodiments, the liquid dispensing robot may include a 6 degrees-of-freedom (DOF) locomotion module. The locomotion module enables displacement in each of the three-dimensions (X, Y, Z) allowing a position of the liquid dispensing robot to be controllably varied. In addition, the locomotion module also enables rotation or angular displacement in each of the three-dimensions (X, Y, Z) allowing an orientation of the liquid dispensing robot to be controllably varied. As such, a “pose of the liquid dispensing robot”, which comprises both a position and an orientation of the liquid dispensing robot may be controlled according to the drug delivery plan.

In various embodiments, the locomotion module of the liquid dispensing robot may also be magnetically activated. The locomotion module may also be controlled by the control magnetic field with a locomotion frequency which is different and non-overlapping with the dispensing frequencies. The locomotion frequency may be a quasistatic frequency. As an example, the locomotion frequency may be 8 Hz or lower. Therefore, synergy arises in the process of drug dispensing and locomotion, both of which are controllable under a single control magnetic field with the varying of operational frequency. Therefore, the process of drug dispensing and/or locomotion may be collectively performed under the control magnetic field.

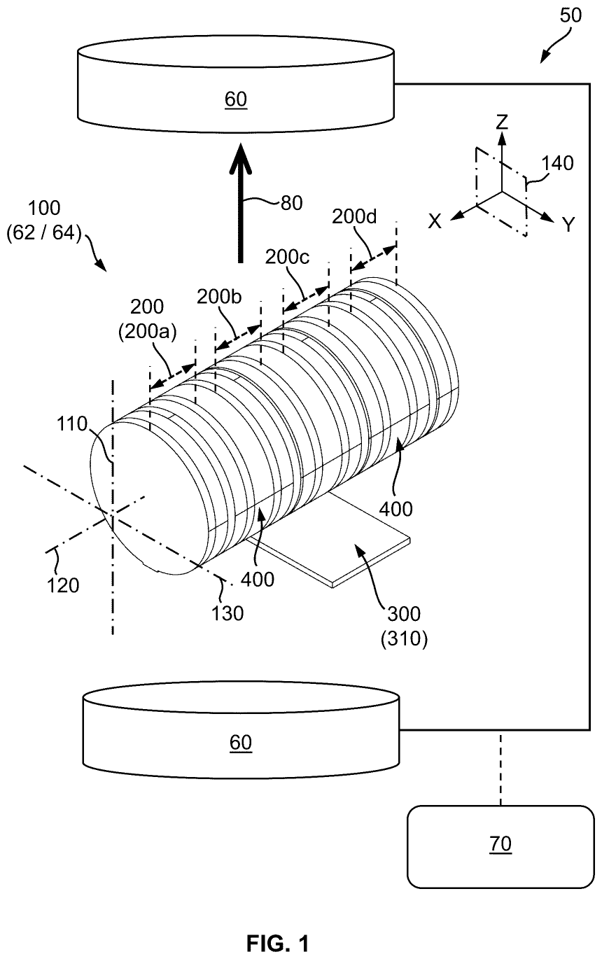

FIG. 1 illustrates a liquid dispensing robotic system 50 for selectively dispensing multiple liquids according to various embodiments of the present disclosure. The multiple liquids may be liquid-based drugs for use in medicinal drug delivery. When used for drug delivery, the liquid dispensing robotic system 50 may be an in-vivo liquid drug delivery system configured for delivering drugs from within the human body. In some examples, the multiple liquid-based drugs may be drugs of different chemical form, medicinal nature or therapeutic purpose. In other examples, the liquid-based drugs may be similar drugs of different concentration and/or dosage. In yet other examples, the liquid-based drugs may be a combination of drugs and other liquids for complimentary purpose to the drugs.

The liquid dispensing robotic system 50 may comprise: an electromagnetic coil system 60 defining a workspace 62. The electromagnetic coil system 60 may be controlled by a controller 70 for providing a control magnetic field 80 to the workspace 62. In various embodiments, the control magnetic field 80 may be an alternating magnetic field with one or more magnetic frequencies. The control magnetic field 80 may be an alternating magnetic field with one or more magnetic frequency bands. Each of the magnetic frequency bands may be non-overlapping and independent from one another. In some embodiments, the control magnetic field 80 may be a quasi-static magnetic field. In some embodiments, the control magnetic field 80 may be a combination or superposition of an alternating magnetic field and a quasi-static magnetic field.

The liquid dispensing robotic system 50 may further comprise: a liquid dispensing robot 100 disposed in the workspace 62. The liquid dispensing robot 100 may comprise a plurality of dispensing modules 200. Each dispensing module 200 may be magnetically actuatable or magnetically controllable. According to an example shown in FIG. 1, the liquid dispensing robot 100 may include four dispensing modules 200a/200b/200c/200d. The number of dispensing modules 200 may be varied according to the drug delivery requirements.

In various embodiments, the liquid dispensing robot 100 may further include a locomotion module 300. The locomotion module 300 enables the liquid dispensing robot 100 to perform locomotor activities, such as performing linear displacement to vary a position of the liquid dispensing robot 100 and/or performing angular displacement to vary an orientation of the liquid dispensing robot 100.

In various embodiments, the liquid dispensing robot 100 may define a magnetic axis 110. The magnetic axis 110 may be in alignment with a net magnetic moment of the liquid dispensing robot 100. It may be noted that the net magnetic moment is a combination of magnetic moments from each of the components in the liquid dispensing robot 100. In various embodiments, a magnetization profile of the liquid dispensing robot 100 may be symmetrical about the magnetic axis 110.

In some embodiments, the plurality of dispensing modules 200 may be disposed adjacent to one another along a longitudinal axis 120, wherein the longitudinal axis 120 is orthogonal to the magnetic axis 110. This allows the size of the liquid dispensing robot 100 to be kept compact.

In various embodiments, each of the plurality of dispensing modules 200 may hold a liquid-based drug fluidly separated or insulated from other ones of the plurality of dispensing modules 200. In other words, the liquid-based drugs held in each of the dispensing module 200 is separated from each other. In various embodiments, each of the dispensing module 200 may be separated by a respective spacer 400.

In various embodiments, each of the plurality of dispensing modules 200 may define a respective actuation frequency band. Each of the plurality of dispensing modules 200 may be activated to dispense the held liquid-based drug when the control magnetic field has a frequency within the respective actuation frequency band. In some examples, the actuation frequency band may be a narrow band, such as 20 Hz to 21 Hz. In response to the control magnetic field 80 with a dispensing frequency within the actuation frequency band of the dispensing module 200, the corresponding dispensing module 200 will dispense the liquid-based drug held in the dispensing module 200. In some examples, the actuation frequency band may include only a single frequency value.

In various embodiments, the actuation frequency bands of the plurality of dispensing modules 200 may be non-overlapping or distinct from one another, to allow each of the dispensing module 200 to be independently controlled. In some embodiments, the actuation frequency bands may comprise a resonance frequency (or natural frequency) of the respective dispensing module 200. For example, the actuation frequency band for dispensing module 200a is in a range of 26 Hz to 27 Hz, which is non-overlapping and distinct from the actuation frequency band for dispensing module 200b, which is in a range of 61 Hz to 62 Hz. Therefore, when a control magnetic field 80 with a frequency of 61.5 Hz is applied to the liquid dispensing robot 100, the liquid-based drug held in dispensing module 200b will be dispensed. As such, each of the plurality of dispensing modules 200 may be selectively and independently controlled by the control magnetic field 80 to dispense the liquid-based drug from the respective dispensing module 200.

In various embodiments, the locomotion module 300 may include a pair of deformable tentacles 310 or legs. The pair of tentacles 310 may be magnetized. The pair of tentacles 310 may be controlled or displaced responsive to the control magnetic field 80 with a locomotion frequency which is different and non-overlapping with each of the actuation frequency bands of the plurality of dispensing modules 200. The locomotion frequency may be a quasistatic frequency. The pair of tentacles 310 may be symmetrically arranged about the magnetic axis 110. The pair of tentacles 310 may define a magnetic moment symmetrical about the magnetic axis 110. In various embodiments, the pair of tentacles 310 may extend along a lateral axis 130, which is orthogonal to both the longitudinal axis 120 and the magnetic axis 110.

In various embodiments, the plurality of dispensing modules 200 and the locomotion module 300 collectively defines a net magnetic moment aligned with the magnetic axis 110. In various embodiments, the plurality of dispensing modules 200 and the locomotion module 300 collectively defines a net magnetic moment parallel to the magnetic axis 110.

In addition, each of the plurality of dispensing modules 200 may be configured in a substantially planar form parallel to a front plane 140, the front plane defined by the magnetic axis 110 and the lateral axis 130.

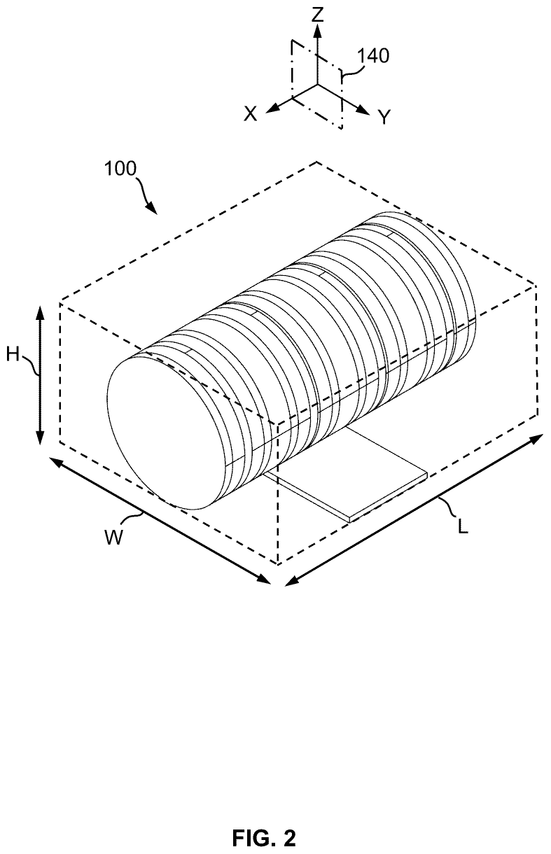

Further referring to FIG. 2, the liquid dispensing robot 100 may be a miniature robot diametrically defining a length (L), a width (W) and a height (H). In various embodiments, dimensions (L, W, H) of the liquid dispensing robot 100 may be configured based on different applications. In exemplary applications such as for human central nervous system and the ureter/urethra of adult humans, the maximum dimension (L, W, H) of the liquid dispensing robot 100 may be 1.5 millimetres (mm). In other applications such as gastrointestinal tract or colorectal cancer, the maximum dimension (L, W, H) of the liquid dispensing robot 100 may be around 4 millimetres (mm).

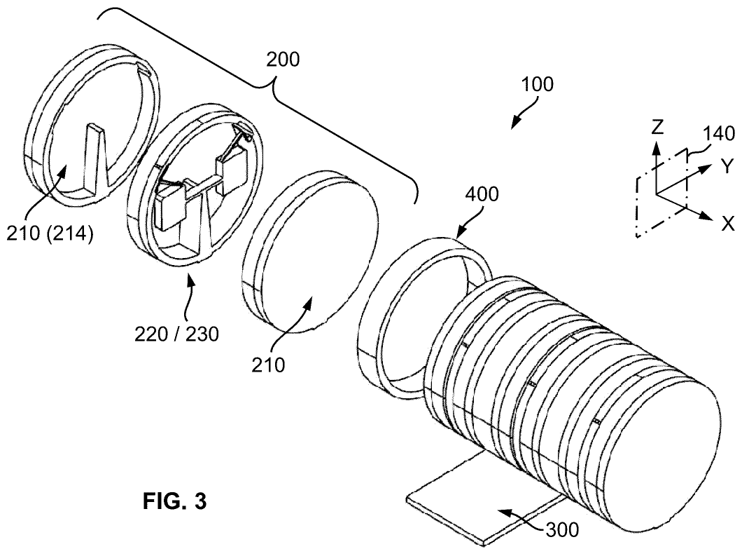

FIGS. 3 to 4 illustrate a dispensing module 200 according to various embodiments of the disclosure. The following description of the exemplary dispensing module 200 may be applicable to selected ones or all of the plurality of dispensing modules 200 of the liquid dispensing robot 100.

According to various embodiments, the dispensing module 200 may include a housing 210 defining an inner chamber 214. The inner chamber 214 may define a volume for holding a first liquid, such as a liquid-based drug. The housing 210 may be formed from multiple parts or may be integrally formed, for example with additive manufacturing. The housing 210 may be formed as a passive non-magnetic component of the dispensing module 200. The housing 210 may be made of a biocompatible polymer, such as pristine polydimethylsiloxane (PDMS), which has an elastomer base to curing agent mass ratio of 10:1. The pristine PDMS is known to be biocompatible in nature and hence suitable as a vessel for drug delivery.

In various embodiments, the housing 210 may also define an opening 212. The opening 212 may be formed through a surface or wall of the housing 210 allowing fluid communication between the inner chamber 214 and an environment 64 exterior of the housing 210. The opening 212 may be sized to provide a fluid resistance such that when the dispensing module 200 is not activated to dispense, the first liquid or the liquid-based drug does not leak from the opening 212 to the environment 64 and may be contained and held within the housing 210. This may be enabled by an equalized fluid pressure between the inner chamber 214 and the environment 64, in combination with friction head loss across the opening 212 for the liquid-based drug. As such, it may be understood that dispensing the liquid-based drug from the inner chamber 214 may be activated in response to a pressure differential formed between the inner chamber 214 or at least a part of the inner chamber 214 and the environment 64, which overcomes the friction head loss or fluid resistance from the opening 212.

In various embodiments, the dispensing module 200 may further include an actuating member 220 and a dispensing member 230 for varying a fluid pressure in the inner chamber 214 or at least a local fluid pressure in the inner chamber 214. The local fluid pressure may correspond to the liquid-based drug adjacent to the opening 212.

The actuating member 220 may be a magnetized member made by embedding NdFeB microparticles (Magnequench: average size of 5 μm) into a PDMS polymer matrix based on a 1:1 mass ratio. The elastomer base and curing agent of this PDMS matrix may have a corresponding mass ratio of 7:1. The dispensing member 230 may be a non-magnetized member made of a biocompatible polymer, such as pristine polydimethylsiloxane (PDMS), which has an elastomer base to curing agent mass ratio of 10:1.

In various embodiments, the actuating member 220 may be coupled to the housing 210 disposed in the inner chamber 214. The actuating member 220 may be movable or at least in part displaceable relative to the housing 210. The actuating member 220 may define an actuation frequency band. The actuating member 220 may be magnetically actuated responsive to a magnetic field with a dispensing frequency (B_{A}) within the actuation frequency band. In some examples, the actuation frequency band may correspond to a range of frequencies comprising a resonance frequency of the actuating member 220. In other examples, the actuation frequency band may correspond to a range of frequencies which enables sufficient deformation or displacement to the actuating member 220 for drug dispensing.

In various embodiments, the dispensing member 230 may also be coupled to the housing 210 and disposed in the inner chamber 214 adjacent to the opening 212. The dispensing member 230 may be spaced apart from the opening 212. The dispensing member 230 may be movable or at least in part displaceable relative to the housing 210. The dispensing member 230 may be movable responsive to deformation or displacement of the actuating member 220. The actuating member 220 may displace or deform to periodically displace the dispensing member 230 relative to the opening 212 to vary a local fluid pressure adjacent to the opening 212. In other words, the actuating member 220 may cause the dispensing member 230 to oscillate or to be in oscillation. Displacement of the dispensing member 230 may vary or increase the local fluid pressure of the liquid-based drug adjacent to the opening 212. The increase in the local fluid pressure may be sufficient to overcome the friction head loss of the opening 212, causing the liquid-based drug to be dispensed from the inner chamber 214 to the environment 64.

In an exemplary embodiment, the actuating member 220 may be deformable or movable by the control magnetic field 80, thus causing the actuating member 220 to be in physical contact with the dispensing member 230, directly displacing the dispensing member 230 relative to the housing 210. In alternative embodiments, the actuating member 220 may, for example by use of a connecting element, indirectly displace the dispensing member 230 relative to the housing 210. In yet other alternative embodiments, the actuating member 220 may be fixedly coupled or integrally formed with the dispensing member 230 such that deformation of the actuating member 220 displaces the dispensing member 230 relative to the housing 210.

Referring again to the liquid dispensing robot 100 of FIG. 1, the respective actuation frequency bands of the plurality of actuating members 220 are configured to be non-overlapping with and distinct from one another. This enables each of the actuating member 220 of the dispensing modules 200 to be selectively actuated or stopped by the control magnetic field 80 with a dispensing frequency (B_{A}). A contrary to the non-overlapping actuation frequency band introduces a risk of unavoidably dispensing liquid-based drugs from two or more dispensing modules 200 concurrently.

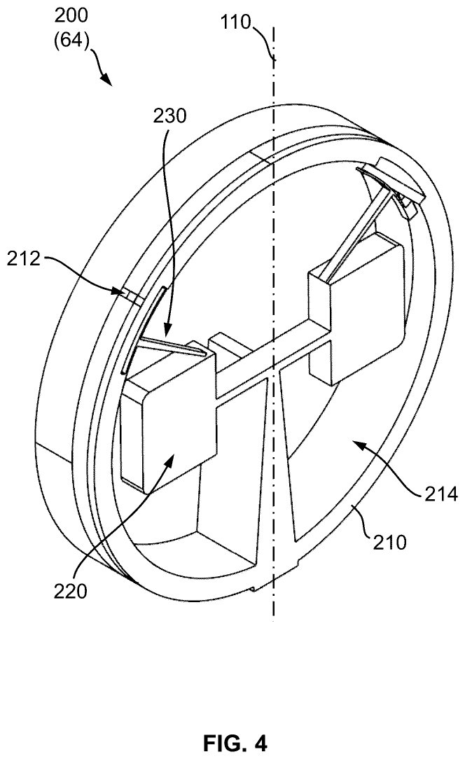

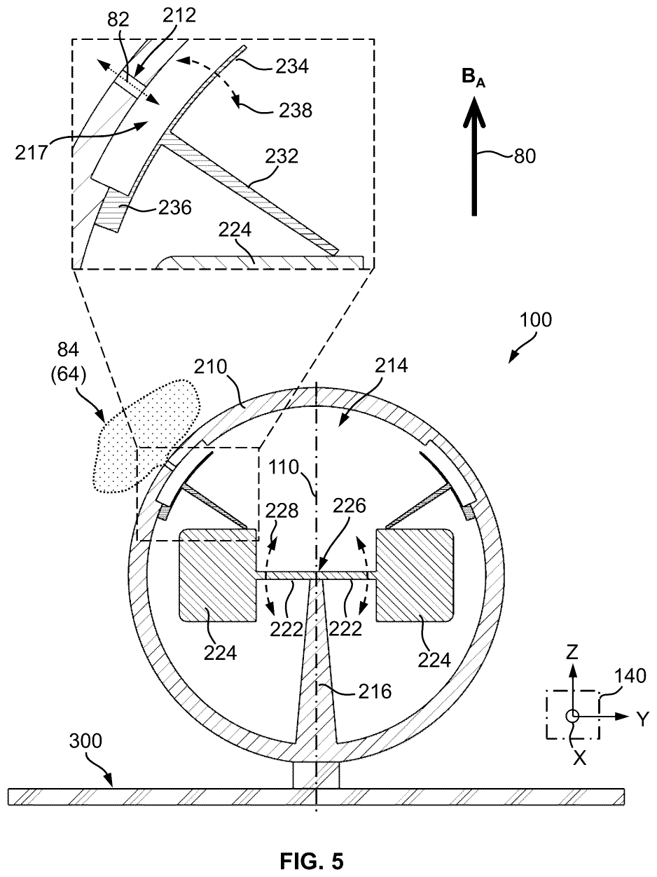

FIGS. 5 to 6C illustrate a dispensing module 200 according to various embodiments of the disclosure. The dispensing module 200 may include a housing 210 defining an inner chamber 214. A first liquid or a liquid-based drug may be held in the inner chamber 214. The housing 210 may include an opening 212 forming a fluid communication between the inner chamber 214 and an environment 64. The opening 212 enables fluid pressure equalization between the inner chamber 214 and the environment 64 in the course of dispensing the liquid-based drugs. The dispensing module 200 may further include an actuating member 220 and a dispensing member 230, both of which are coupled to the housing 210 and disposed in the inner chamber 214.

In various embodiments, the actuating member 220 may include a pair of beams 222 with a corresponding mass 224 fixedly coupled to each end of the pair of beams 222. A connecting portion or interface between the pair of beams 222 may act as a coupling portion 226 for coupling to a support 216 of the housing 210. The support 216 may be formed extending into the inner chamber 214. The pair of beams 222 may each be fixedly coupled to the housing 210 via a fixed coupling between the coupling portion 226 and the support 216. As such, the pair of beams 222 may form a pair of cantilever beams 222 about the coupling portion 226/support 216 with the mass 224 defining each end of the pair of cantilever beams 222. The actuating member 220 may be symmetrical about the magnetic axis 110 of the liquid dispensing robot 100.

In various embodiments, each of the mass 224 may be a magnetized mass, or in other words, the mass 224 may be a magnetic body or a magnetized member with a magnetic moment. Responsive to a control magnetic field 80, the masses 224 may align with the control magnetic field 80 causing a deformation and displacement to the pair of beams 222. Alignment of the masses 224 with the control magnetic field 80 deforms or displaces the actuating member 220 relative to the housing 210. It may be understood that under an alternating control magnetic field 80, the pair of beams 222 may displace in a cyclic or periodic oscillating motion 228.

The actuating member 220 may define an actuation frequency band. The actuation frequency band may be mechanical in nature. The actuation frequency band may correspond to a resonating and oscillating motion of the actuating member 220. The actuation frequency band may correspond to a range of frequencies which comprises a resonance frequency of the actuating member 220. In other examples, the actuation frequency band may correspond to a range of frequencies which enables sufficient deformation or displacement to the actuating member 220 for drug dispensing. Hence, the magnitude of the displacement or oscillation of the actuating member 220 may depend on the dispensing frequency (B_{A}) of the control magnetic field 80 and whether the dispensing frequency (B_{A}) is within the actuation frequency band.

In various embodiments, the actuation frequency band of the actuating member 220 may be determined by various mechanical parameters such as a thickness of each beam 222, a length of each beam 222, an elastic property of each beam 222, the weight of the mass 224, a magnetic moment of the mass 224, etc.

Similar to the actuating member 220, the dispensing member 230 may also be coupled to the housing 210 and disposed in the inner chamber 214. The dispensing member 230 may be movable or at least in part displaceable relative to the housing 210. The dispensing member 230 may be movable responsive to deformation or displacement of the actuating member 220. In other words, displacement of the actuating member 220 causes a corresponding displacement of the dispensing member 230. Similarly, oscillation of the actuating member 220 causes a corresponding oscillation of the dispensing member 230. The dispensing member 230 may be formed as a passive non-magnetic component of the dispensing module 200. The dispensing member 230 may also be symmetrical about the magnetic axis 110 of the liquid dispensing robot 100.

In various embodiments, the dispensing member 230 may include a contact portion 232 for contacting the actuating member 220. The dispensing member 230 may further include a flap portion 234 adjacent to the opening 212. The flap portion 234 may be spaced apart from the opening 212 to define the adjacent zone 217. A fixing end 236 of the dispensing member 230 may be fixedly coupled to the housing 210 such that the dispensing member 230 may also form a cantilever. In some alternative embodiments, the fixing end 236 and the dispensing member 230 may be integrally formed with the housing 210. As the dispensing member 230 is a passive non-magnetized member, displacement of the dispensing member 230 may be driven by the actuating member 220. In various embodiments, displacement of the dispensing member 230 is caused by physical contact between the contact portion 232 of the dispensing member 230 and the mass 224 of the actuating member 220.

In various embodiments, the dispensing member 230 may at least be partially displaceable along a liquid flow axis 82 through the opening 212. The liquid flow axis 82 may be defined by the opening 212. The displacement or oscillating motion 228 of the actuating member 220 may periodically displace 238 or periodically deform the dispensing member 230. This causes the flap portion 234 to displace relative to the opening 212. In various embodiments, the flap portion 234 may be at least partially displaceable along the liquid flow axis 82.

FIGS. 6A to 6C illustrate the oscillating motion of the actuating member 220 and the dispensing member 230. Referring to FIG. 6A, the periodic displacement of the flap portion 234 increases the local fluid pressure of the liquid-based drug disposed in an adjacent zone 217. The adjacent zone 217 may be neighbouring the opening 212, and thus the first liquid (for example the liquid-based drug) is dispensed from the inner chamber 214 to the environment 64. The liquid-based drug may exit the adjacent zone 217 of the inner chamber 214 through the opening 212 along the liquid flow axis 82. FIG. 6B shows the actuating member 220 and the dispensing member 230 in a neutral position.

Further referring to FIG. 6C, in addition to the liquid-based drug being dispensed 84 through the opening 212, periodically displacing the dispensing member 230 may also cause a second liquid (for example saline or bodily fluid) in the environment 64 to enter the inner chamber 214 through the opening 212 along the liquid flow axis 82. Therefore, the dispensing action of the dispensing module 200 comprises expelling liquid-based drug from the inner chamber 214 and receiving bodily fluid from the environment 64 such that the inner chamber 214 and the environment 64 achieve fluid pressure equalization. In various embodiments, due to the opening 212 sized to provide the corresponding fluid resistance, the first liquid or liquid-based drug may be contained or held within the inner chamber 214 in the absence of the displacement of the dispensing member 230. In addition, the first liquid or liquid-based drug also may be contained or held within the inner chamber 214 in the absence of sufficient displacement of the dispensing member 230.

In various embodiments, the electromagnetic coil system 60 may provide a control magnetic field 80 with a magnetic frequency corresponding to a dispensing frequency (B_{A}). The dispensing frequency (B_{A}) is within the actuation frequency band. In response to the control magnetic field 80 with the dispensing frequency (B_{A}), the actuating member 220 may deform and displace (or oscillate) relative to the housing 210. Further, when subjected to the control magnetic field 80 with the dispensing frequency (B_{A}), the actuating member 220 may displace (or oscillate) sufficiently to displace the dispensing member 230 relative to the opening 212 to increase the local fluid pressure of the liquid-based drug adjacent to the opening 212. Hence, when the increase in the local fluid pressure causes the local fluid pressure to exceed a threshold pressure, the local fluid pressure overcomes the resistance from the opening 212 (as previously described) causing the liquid-based drug in the inner chamber 214 to be dispensed 84 through the opening 212 from the inner chamber 212 to the environment 64.

It may be appreciated that while the actuating member 220 may still be displaced by the control magnetic field 80 at a magnetic frequency outside of the actuation frequency band, the displacement of the actuating member 220 may be insufficient to displace the dispensing member 230 in such a way to cause the local fluid pressure to exceed a threshold pressure. This may be due to the magnetic frequency being too far from a fundamental natural frequency of the actuating member 220, thus producing limited displacement or oscillation (if any). Hence, dispensing of the liquid-based drug cannot be achieved when the magnetic frequency is outside of the actuation frequency band. This reduces the risk of leakage during operation.

Referring again to FIG. 1, as each of the plurality of dispensing modules 200 defines a non-overlapping and distinct actuation frequency band, the liquid dispensing robot 100 may selectively dispense the respective liquid-based drug from the selected dispensing module 200 by controlling the dispensing frequency (B_{A}) of the control magnetic field 80. It may be noted that when it is desired for more than one liquid-based drug to be dispensed concurrently or at least partially concurrently, the dispensing frequency (B_{A}) may include more than one magnetic frequency, each magnetic frequency within a respective actuation frequency band.

In various embodiments, when the magnetic frequency of the control magnetic field 80 is outside of the actuation frequency band of the actuating member 220, the actuating member 220 may be spaced apart from and not in physical contact with the dispensing member 230 in the inner chamber 214. In these embodiments, the actuating member 220 may only contact the dispensing member 230 when the magnetic frequency of the control magnetic field 80 is within the actuation frequency band of the actuating member 220. This ensures that the dispensing member 230 remains stationary and is not in motion, reducing the risk of unintended drug leakage.

In various embodiments, when the magnetic frequency of the control magnetic field 80 is outside of the actuation frequency band of the actuating member 220, the actuating member 220 may still be in contact with the dispensing member 230 to displace or deform the dispensing member 230. However, the displacement or deforming of the dispensing member 230 may be limited or reduced, and thus does not increase the local fluid pressure sufficiently to overcome the friction head loss or resistance of the opening 212.

Referring now to FIG. 7, in various embodiments of the dispensing module 200, the housing 210 may include multiple openings 212. Therefore, the liquid-based drug held in the inner chamber 214 may be dispensed 84 to the environment 64 through the multiple openings 212 along the respective liquid flow axis 82. As shown in FIG. 7, as the openings 212 are symmetrically positioned about the magnetic axis 110, the liquid dispensing robot 100 may be symmetrical about the magnetic axis 110.

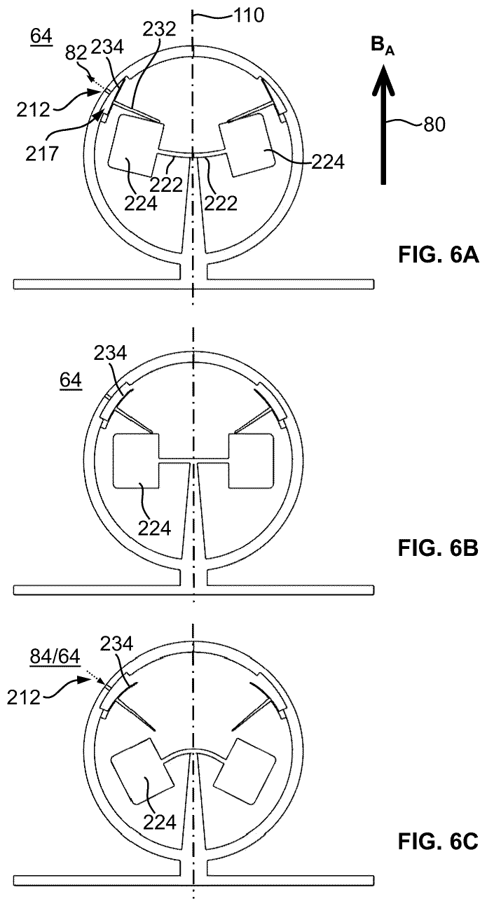

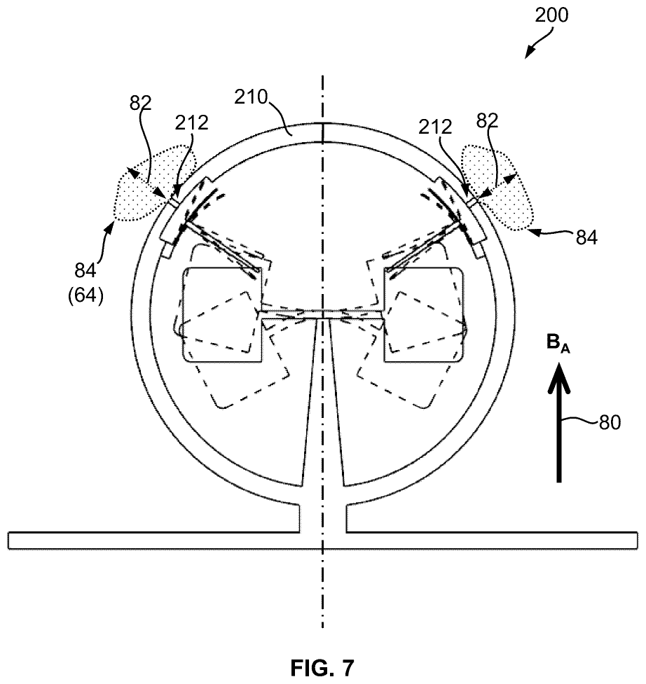

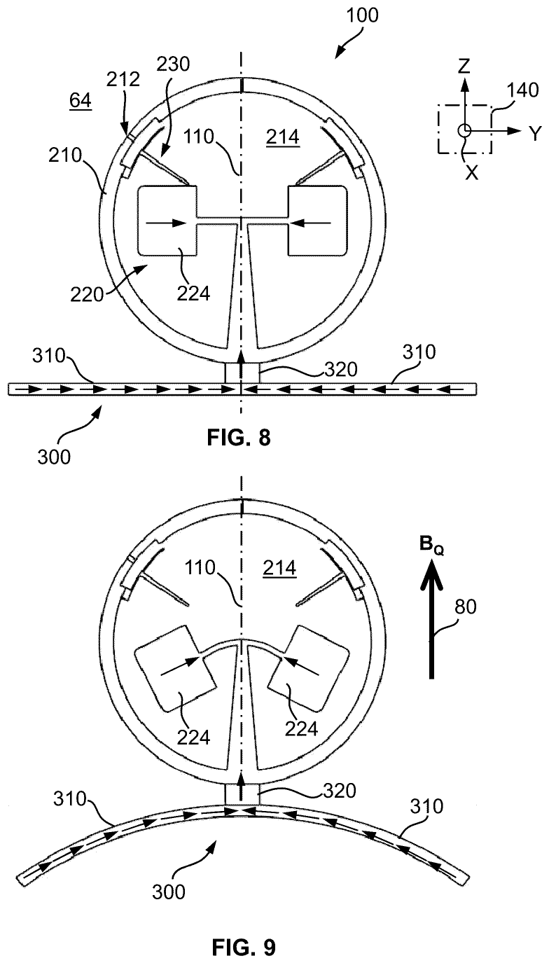

FIGS. 8 and 9 are sectional views of a liquid dispensing robot 100 according to various embodiments. The sectional view shows one dispensing module 200 with the locomotion module 300. It may be understood that the other dispensing modules 200 are of similar configuration.

The dispensing module 200 comprises a housing 210 defining an opening 212 and an inner chamber 214. The opening 212 enables fluid communication between the inner chamber 214 and an environment 64. The dispensing module 200 further comprises an actuating member 220 and a dispensing member 230. The actuating member 220 may comprise a pair of magnetized mass 224 disposed on opposing sides of the actuating member 220.

In addition, the locomotion module 300 may include a pair of tentacles 310 and an auxiliary magnet 320 arranged along the magnetic axis 110. The locomotion module 300 may define a locomotion magnetic dipole aligned with or parallel to a magnetic axis 110 of the robot. The auxiliary magnet 320 may provide a general magnetic moment to the liquid dispensing robot 100. The pair of soft tentacles 310 may be made by embedding the PDMS polymer matrix (Dow Corning) with NdFeB magnetic microparticles (Magnequench: average size of 5 μm). The mass ratio between the NdFeB magnetic microparticles and PDMS may be 3:1, while the elastomer base and curing agent of the PDMS may have a mass ratio of 7:1. The auxiliary magnet 320 may be made by embedding NdFeB microparticles (Magnequench: average size of 5 μm) into a PDMS polymer matrix based on a 1:1 mass ratio. The elastomer base and curing agent of this PDMS matrix may have a corresponding mass ratio of 7:1.

The arrows in FIGS. 8 and 9 show the magnetic moment of the masses 224, the pair of tentacles 310 and the auxiliary magnet 320.

Referring to FIG. 8, when the liquid dispensing robot 100 is in a neutral state or inactivated state, the magnetic moments of the two masses 224 are symmetrical about the magnetic axis 110. Similarly, the magnetic moments of the locomotion module 300 is also symmetrical about the magnetic axis 110. Hence, a net magnetic moment of the liquid dispensing robot 100 is also symmetrical about the magnetic axis 110.

FIG. 9 illustrates the liquid dispensing robot 100 when subjected to a control magnetic field 80 with a locomotion frequency (B_{Q}), such as a quasistatic frequency, to control a locomotion activity of the liquid dispensing robot 100. The pair of tentacles 310 may deform or displace responsive to the control magnetic field 80 for locomotion. It may be noted that the locomotion frequency (B_{Q}) may be non-overlapping and distinct from each of the actuation frequency bands. Similar to the neutral state, under the control magnetic field 80 with the locomotion frequency (B_{Q}), the magnetic moments of the two masses 224 as well as the pair of tentacles 310 are symmetrical about the magnetic axis 110.

In various embodiments, the masses 224 of the actuating member 220 is biased away from the dispensing member 230 responsive to the control magnetic field 80 with the locomotion frequency (B_{Q}). It may be noted that responsive to the control magnetic field 80 with the locomotion frequency (B_{Q}), the masses 224 move away from the dispensing member 230, thus reducing the risk of accidental contact with the dispensing member 230 during pure locomotion activity.

In various embodiments, it may be appreciated that the control magnetic field 80 may include a dispensing frequency (B_{A}) as well as a locomotion frequency (B_{Q}). This allows the liquid dispensing robot 100 to perform locomotion as well as dispensing of drugs concurrently. In other words, the liquid dispensing robot 100 may dispense the liquid-based drugs on the move.

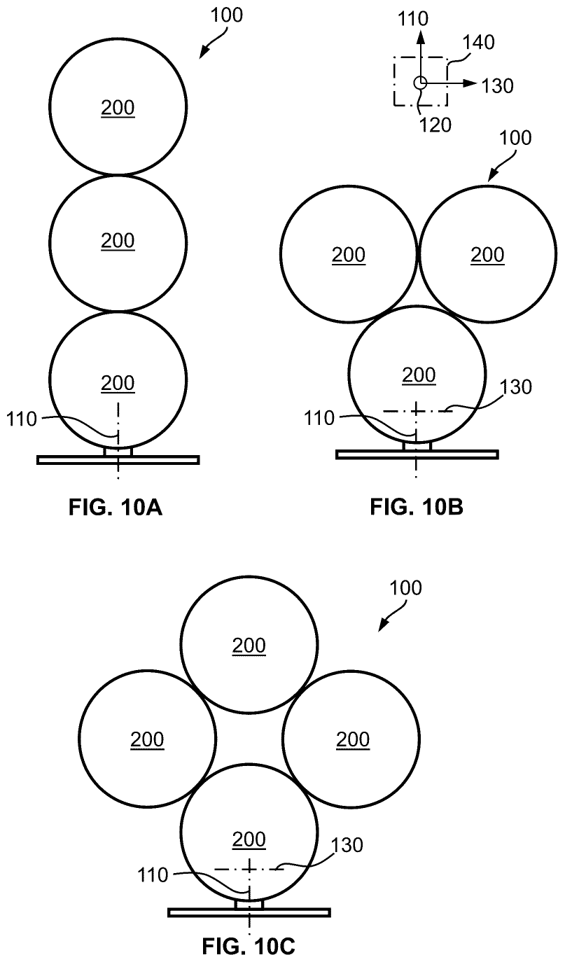

FIGS. 10A to 10C illustrate various exemplary embodiments of the liquid dispensing robot 100. As shown in FIG. 10A, in some embodiments, the plurality of dispensing modules 200 may be arranged along the magnetic axis 110. In other embodiments, the plurality of dispensing modules 200 may be arranged along both the magnetic axis 110 and the lateral axis 130.

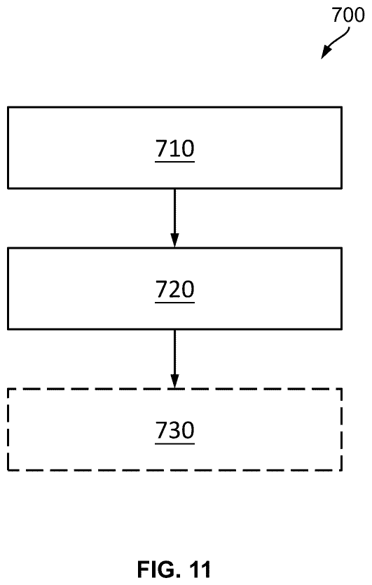

According to another aspect, as shown in FIG. 11, disclosed herein a method 700 of liquid dispensing. The method 700 comprises: in 710, providing a control magnetic field with a first dispensing frequency to the liquid dispensing robot as described above; and in 720, providing the control magnetic field with a second dispensing frequency to the liquid dispensing robot, wherein the first dispensing frequency is within a first actuation frequency band and the second dispensing frequency is within a second actuation frequency band, wherein the first actuation frequency band is non-overlapping with the second actuation frequency band.

In various embodiments, the first dispensing frequency and the second dispensing frequency of the control magnetic field may be independently provided non-overlapping in time.

In various embodiments, the method 700 may further comprise: providing the control magnetic field with a locomotion frequency to move the liquid dispensing robot to a target position, the locomotion frequency non-overlapping with each of the first actuation frequency band and the second actuation frequency band. In various embodiments, the control magnetic field comprises at least one of: the first dispensing frequency and the second dispensing frequency provided at least partially concurrent with the locomotion frequency.

### Exemplary Embodiment

As an exemplary embodiment of the liquid dispensing robot, disclosed herein a millimeter-scale liquid dispensing robot or soft robot that can be magnetically actuated to transport four types of drugs in which their drug-dispensing sequence and dosage can be reprogrammed upon command at multiple locations. The proposed robot is constructed by a combination of smart magnetic composites. To evaluate the biocompatibility of these smart magnetic composites, cytotoxicity experiments were conducted. The anonymous human dermal fibroblast cells (ATCC) that are used in these cytotoxicity experiments show that 98.791-99.633% of these cells remained viable after they are immersed for 24 hours in a Dulbecco's modified eagle medium (DMEM), which previously contains the cuboid samples of the smart magnetic composites (sample dimensions: 5 mm×5 mm×5 mm). Since these viability rates are higher than 98% and they are similar to the viability rate of a control group which is evaluated to be 99.688%, such results suggest that the smart magnetic composites do not cause observable cell damage or death and are biocompatible. Another notable safety feature of the proposed soft robot is that its actuating magnetic fields (\vec{B}) are constrained to be harmless when they penetrate through the human body.

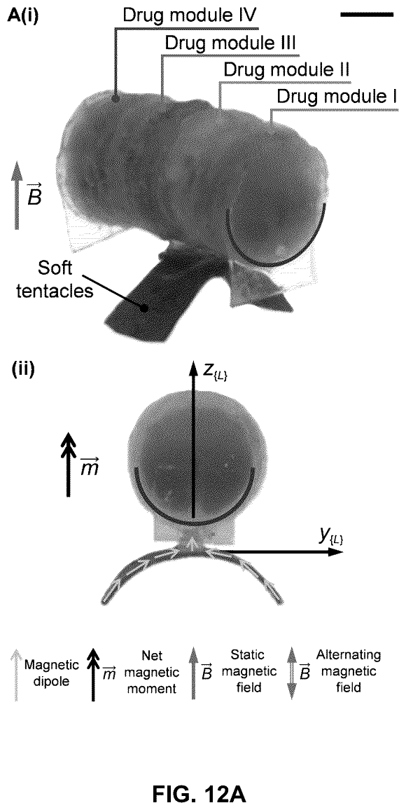

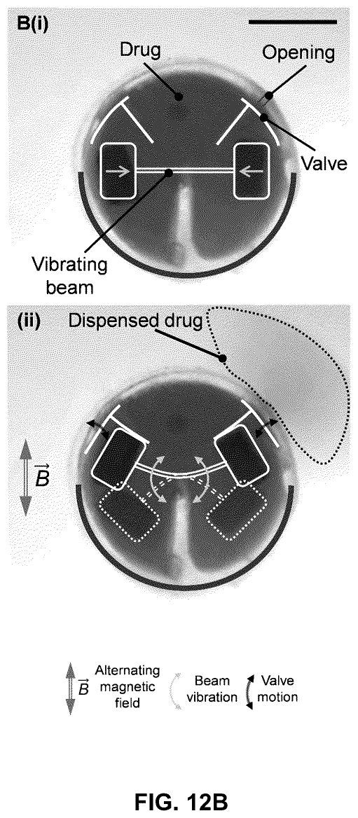

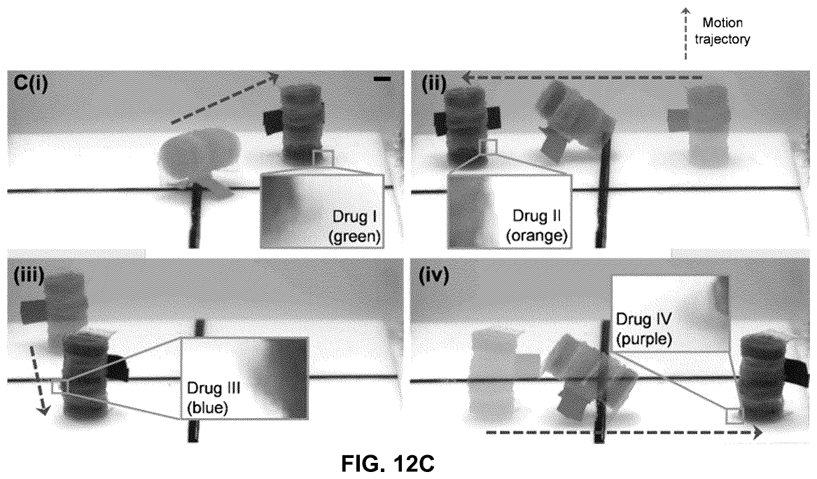

The proposed robot has four similar but non-identical drug modules, an auxiliary magnet, and a pair of soft tentacles (as shown in FIG. 12A). Each drug module carries a unique drug, i.e., drugs I-IV. Within each drug module, there is also a pair of magnetized beams that will vibrate upon command (FIG. 12B). Because the vibrating beams across adjacent drug modules are spaced 1.34 mm apart from one another, there is no observable interaction between these beams regardless of the presence of external magnetic fields. The vibrating beams are programmed in such a way that they will strike the valve in the module to eject the drugs out if and only if we apply a designated, alternating step \vec{B} of selected frequency (f:20-180 Hz) and amplitude (Bₐₘₚ:2-19 mT) (FIG. 12B). Otherwise, the drugs shall remain in the modules. The beams in the drug modules will vibrate under such actuating fields because the magnetic moment at their free ends will always tend to align with the external \vec{B} (FIG. 12B). This phenomenon will in turn generate a magnetic torque at the free ends of the beams, deforming the beams and allowing them to vibrate. The stiffness and fundamental natural frequency of the vibrating beams are configured to be distinct across different drug modules so that their frequency responses are unique (Table 1). It may be noted that the secondary natural frequency is also distinct across the drug modules.

TABLE 1
The dimensions, mass at the free end, stiffness and lowest two
natural frequencies of the vibrating beams in drug modules I-IV

| | | | | Mass at | | | |
|---|---|---|---|---|---|---|---|
| | | | | the free | | Fundamental | Second |
| | Length | Width | | end | | natural | natural |
| Drug | L_{b} | w_{b} | Thickness | m₀ | Stiffness | frequency | frequency |
| module | (mm) | (mm) | h_{b} (mm) | (mg) | (nNmrad⁻¹) | (Hz) | (Hz) |
| I | 0.55 | 0.150 | 0.015 | 0.083 | 2.85 | 26.8 | 1,793 |
| II | 0.50 | 0.150 | 0.025 | 0.096 | 14.5 | 61.6 | 3,616 |
| III | 0.40 | 0.150 | 0.035 | 0.124 | 49.8 | 126 | 7,909 |
| IV | 0.35 | 0.150 | 0.050 | 0.138 | 166 | 249 | 14,760 |

Due to these deliberations, once the f of the alternating step \vec{B} is specified to be similar to the fundamental natural frequency of a given pair of vibrating beams, only these selected beams will produce sufficient vibration to eject the drugs when Bₐₘₚ is sufficiently large. To increase the safety aspects of the actuating magnetic fields, f of the alternating step \vec{B} was selected to be always slightly lower than the computed fundamental natural frequency of the beams (Table 1). By controlling the f and Bₐₘₚ of the applied alternating step \vec{B}, it is possible to reprogram the proposed soft robot's drug-dispensing sequence upon command by dictating which drug to be dispensed. To facilitate the subsequent discussions, the applied alternating step \vec{B} shall be referred to as alternating \vec{B} for simplicity purposes.

The function of the auxiliary magnet is to endow the proposed soft robot with a net magnetic moment while the function of the soft tenacles is to allow the robot to perform various locomotive gaits. A critical advantage of the proposed soft robot is that its overall magnetization and stiffness profiles can allow it to possess full six-DOF motions, potentially allowing it to be much more dexterous than existing five-DOF miniature robots that can dispense drugs. Since the locomotion of the proposed soft robot is controlled by magnetic fields of low frequencies (≤8 Hz), it is possible to effectively decouple the robot's locomotion and drug-dispensing functions via controlling the frequency of \vec{B}. This is a highly beneficial feature endowing the robot with drug-dispensing capabilities.

### Results

Because miniature robots are expected to operate in water-based environments during targeted drug delivery, all of the experiments were conducted in liquid reservoirs to simulate such environmental conditions. Unless specified otherwise, water was used as the reservoir (viscosity of water at 25° C.: 890 μPa·s). As the first demonstration of the proposed soft robot, the robot was placed at the center of a substrate, which was divided into four quadrants (FIG. 12C). The robot was then commanded to roll to each quadrant and dispense a unique drug. This demonstration showed that the proposed soft robot can indeed dispense four drugs. Furthermore, by having the ability to dispense its drugs selectively, the proposed soft robot also showed a major improvement over those existing miniature robots. Unlike miniature robots that must release all their drugs simultaneously at one site, the proposed soft robot is also much more robust since it has no such constraints. Because the proposed soft robot can retain its mobility after the drugs are dispensed, it shows considerable enhancement over existing miniature robots that will lose their mobility after the drugs are dispensed.

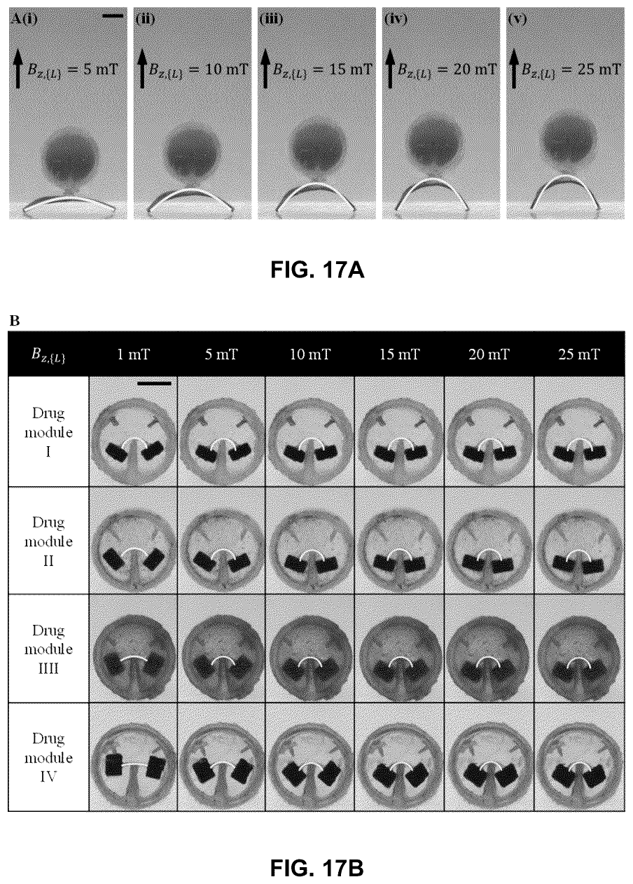

Using magnetic fields of low frequencies (≤2 Hz), the proposed soft robot can be commanded to generate various soft-bodied locomotion like rolling and two-anchor crawling. Of these two gaits, rolling will be a faster mode of locomotion while the two-anchor crawling locomotion can allow the soft robot to position itself more accurately. To describe the motions of the proposed soft robot, a local coordinate frame is attached to the robot body (denoted with a subscript {L} in FIG. 12A (ii). Due to the magnetization profile of the soft robot, its soft tentacles will deform into an inverted “U”-shaped configuration while the drug modules' vibrating beams will also bend away from the valve when a quasistatic \vec{B} is applied along the z_{{L}}-axis. FIGS. 17A and 17B show how the soft robot's tentacles and vibrating beams will bend when the magnitude of \vec{B} is changed from 5 mT to 25 mT. As the vibrating beams and tentacles have a symmetrical arrangement, their deformed configuration will always allow the proposed soft robot's net magnetic moment (\vec{m}) to be parallel to its z_{{L}}-axis regardless of the magnitude of \vec{B}. This is a beneficial feature since it allows the soft robot's \vec{m} to always remain in the same direction under low frequency actuation, allowing a better prediction and thus control of the locomotion of the robot.

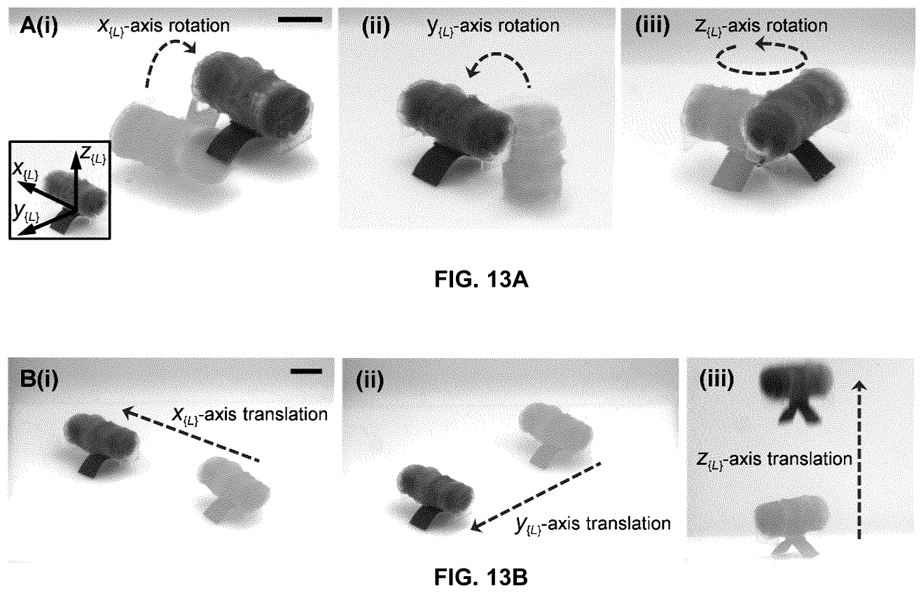

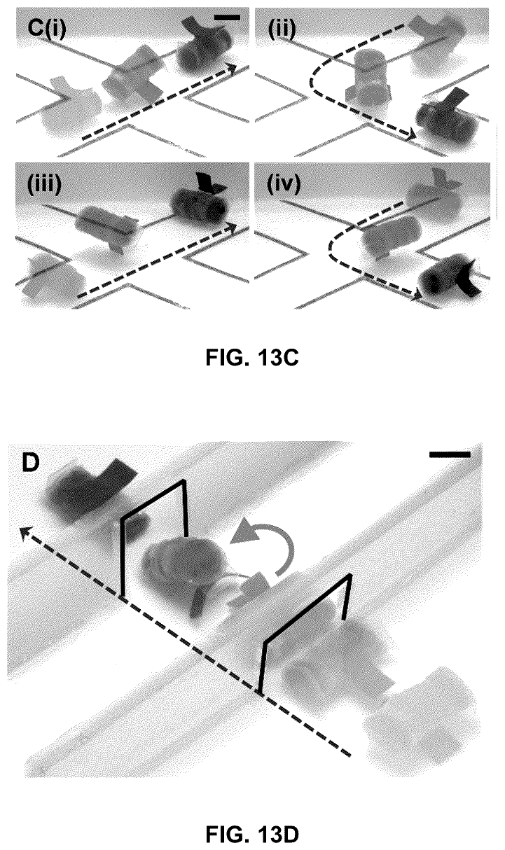

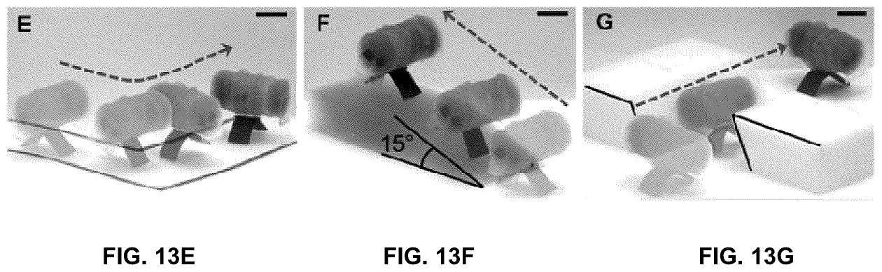

Since the deformed robot's \vec{m} will always tend to align with the applied \vec{B}, it is possible to control the direction of \vec{B} to rotate the soft robot about its x_{{L}}- and y_{{L}}-axes (FIG. 13A(i) and (ii)). By applying appropriate spatial-gradients of \vec{B}, the soft robot may be rotated about its z_{{L}}-axis (the axis about m′ or otherwise known as the six-DOF axis) and translated along three perpendicular axes (FIG. 13A(iii)). In general, the proposed soft robot can achieve linear and angular velocities of 14.7-32.2 mm s⁻¹ and 0.624-47.4 rad s⁻¹, respectively. These velocity measurements are obtained from the experimental videos, where the soft robot's generated linear and angular displacements were divided by the required time taken to produce these corresponding motions. Similar techniques were used for measuring the velocities of the proposed soft robot when it executed other modes of locomotion too and all of the locomotion characterizations are conducted when the soft robot was carrying drugs I-IV. Since the soft robot can produce three independent axes of rotations and translations, this implies that it has six-degrees-of-freedom (DOF) motions. As the dexterity of six-DOF robots is significantly higher than those of their five-DOF counterparts, the proposed soft robot therefore has much greater potential to navigate across the complex, unstructured environments in the human body compared to existing five-DOF robots. Indeed, by having six-DOF, the proposed soft robot can choose to roll along its length or width, and steer the rolling direction (FIGS. 13C and 13D). Having such dexterity empowers the soft robot to roll across challenging obstacles which have strict shape constraints (FIG. 13D). The rolling speeds of the soft robot was also characterized with respect to the frequency of the applied \vec{B}. These experiments indicated that the soft robot can generate speeds of 1.84-16.5 mm s⁻¹ and 1.42-15.2 mm s⁻¹ when it rolls along its length and width at frequencies of 0.1-1 Hz, respectively.

The proposed soft robot can also perform two-anchor crawling gaits by sequentially switching the free-ends of its tentacles as anchors. Similar to the rolling locomotion, the two-anchor crawling speeds of the soft robot was characterized with respect to the frequency of the applied \vec{B}. Using this gait, the soft robot can realize speeds of 0.296-1.34 mm s⁻¹ with frequencies of 0.5-2 Hz. When the proposed soft robot executed the two-anchor crawling gait, it could steer its crawling direction, climb up a slope and negotiate across obstacles with strict shape constraints (FIGS. 13E to 13G). It is noteworthy that existing magnetic miniature robots, which have drug-dispensing capabilities, are unable to accomplish all the tasks in FIGS. 13A to 13G because they only have five-DOF and thus are unable to fully control their orientation. As an example, if the robot does not have control over its z_{{L}}-axis rotation (the sixth-DOF motion), it cannot steer itself to track the paths in FIGS. 13C and 13E when it executes the rolling and two-anchor crawling gaits, respectively. To further highlight the advantages of having six-DOF motions, additional control experiments were performed where one rotational DOF of the proposed soft robot was disabled, constraining it to have five-DOF motions. By having such constraints, the robot will not have sufficient dexterity to roll and two-anchor crawl across the obstacles in FIGS. 13D and 13G, respectively. These experiments further support that the dexterity of the proposed soft robot is indeed significantly higher than its five-DOF counterparts. From the locomotion experiments, a notable observation is that only 4.12-8.08% of the drugs are discharged from the fully drug-loaded robot after it has rolled continuously for 8 hours. The proposed soft robot can minimize its leakage because the vibrating beams in its drug modules are programmed to bend away from the valve during locomotion, thereby minimizing the chances of unintended drug release. By minimizing its drug leakage, the proposed soft robot therefore has great potential to minimize complications when it is deployed for targeted combination therapies in the future.

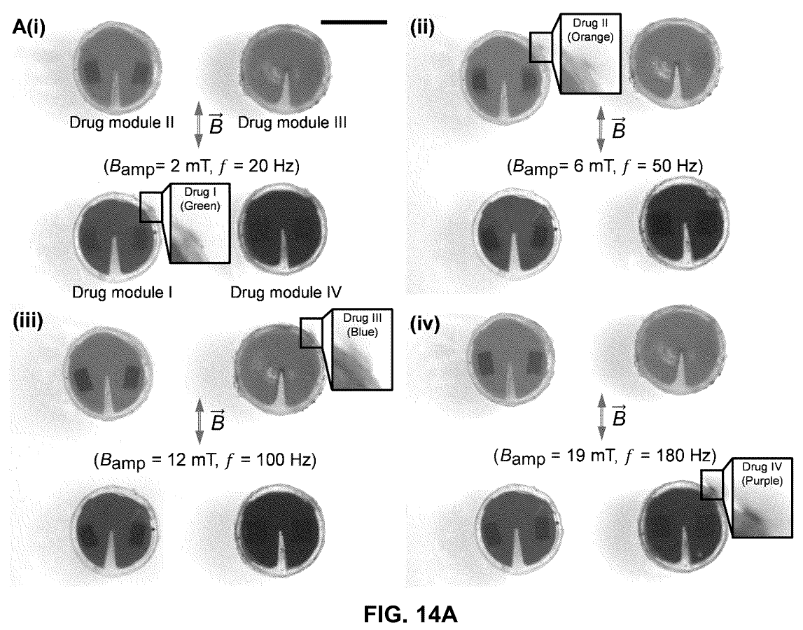

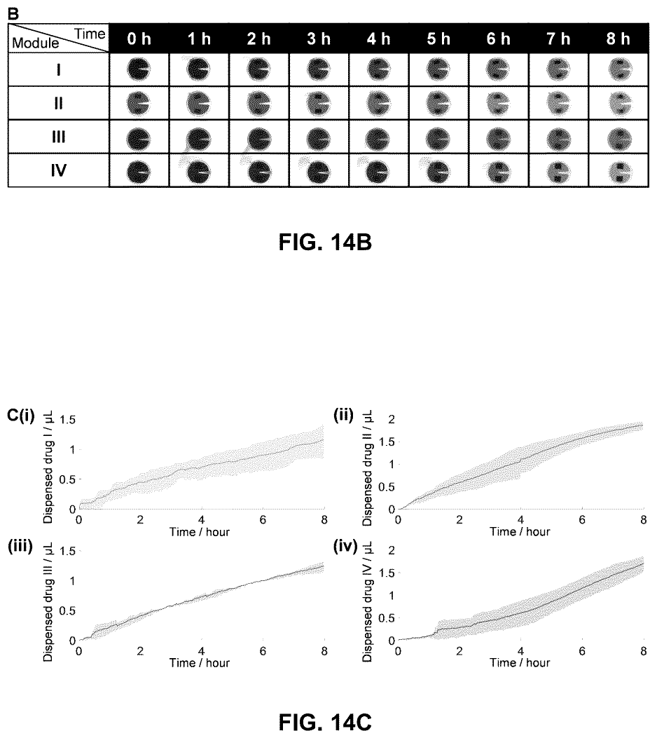

To characterize the proposed soft robot's drug-dispensing performance, additional drug modules were created that are identical to those of the soft robot and were placed on a flat substrate (FIG. 14A). These modules were then actuated by four sets of alternating \vec{B} to evaluate if they can selectively dispense their drugs (FIG. 14A, see later section “Drug-dispensing capabilities”). In these experiments, it showed that the drugs from each module will indeed only be ejected if and only if their respective vibrating beams were subjected to a designated, alternating \vec{B} of selected f and Bₐₘₚ (FIG. 14A). Based on computer vision techniques, the drug-dispensing rate of each module was also measured over a period of 8 hours (FIGS. 14B and 14C). This data is important because it can allow the proposed soft robot to precisely control the drug dosage via regulating the duration of the applied alternating \vec{B}. The experimental results indicated that all the modules could dispense 0.282-0.586 μL of drugs within 2 hours (FIG. 14C), and such dispensing rates are theoretically sufficient for a wide range of combination therapies. The mean dispensing rates for all the drug modules are 0.144-0.231 L/hour across 8 hours (FIG. 14C). Since the medicines used for targeted drug delivery were generally made of colloids that have nanoparticles suspended in water, the simulated drugs I-IV were also similar colloids created by suspending colored nanoparticles in water (see below section “Simulated drugs”). In addition to characterizing the drug-dispensing rate of the robot on flat substrates, experiments were also conducted to show that the proposed soft robot could selectively dispense its drugs on slopes of differing grades (5°-20°) (see later section “Drug-dispensing capabilities”). The drug-dispensing mechanism of the proposed soft robot is robust because its drug modules can remain operational even after their drug contents have changed. However, the drug-dispensing rates of the proposed soft robot may vary when it is loaded with different drugs (see later section “Drug-dispensing capabilities”).

### Simulated Drugs

Because the typical medicine for targeted drug delivery is generally made of nanoparticles (diameter: 1-1000 nm) which are suspended in water, previous experiments were performed using simulated drugs that are created via dissolving colored nanoparticles into water: drug I (green nanoparticles solute: Pilot Pen Corporation), drug II (orange nanoparticles solute: Pilot Pen Corporation), drug III (blue nanoparticles solute: Polysciences Inc), and drug IV (purple nanoparticles solute: Pilot Pen Corporation). To ensure that all of these particles are smaller than 1 μm, experiments on the simulated drugs were conducted based on the Tyndall effect. In this experiment, a laser beam of 635 nm wavelength (Fluke infrared thermometer) is emitted across the simulated drugs stored within a glass chamber. Because the laser can successfully shine across the transparent chamber, such observations imply that the simulated drugs are colloids, which have suspending particles that have diameters of 1-1000 nm. These experiments therefore conclude that drugs I-IV as used in the proposed soft robot are indeed suspended with nanoparticles, which are comparable in size to those of the clinical drugs.

### Fluorescent Dyes

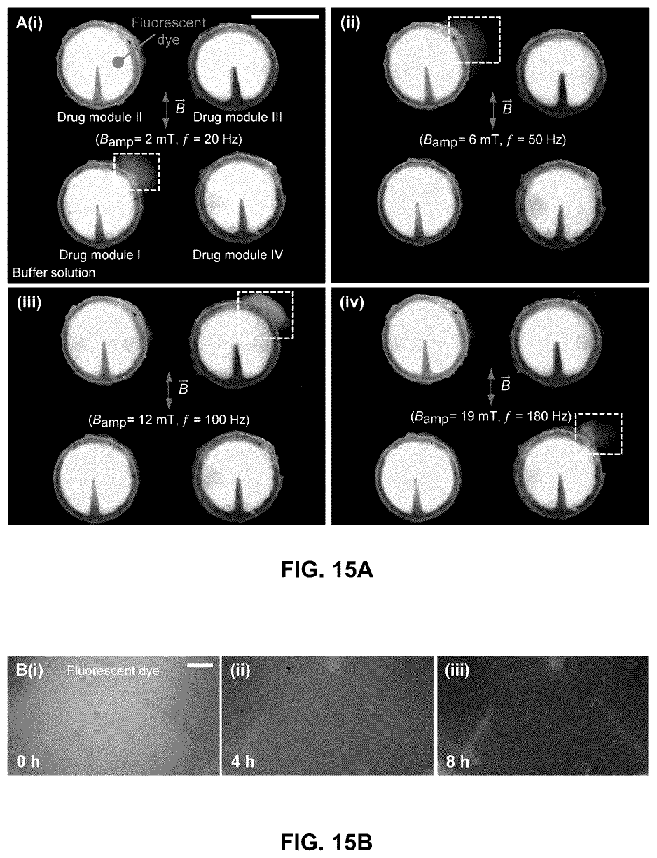

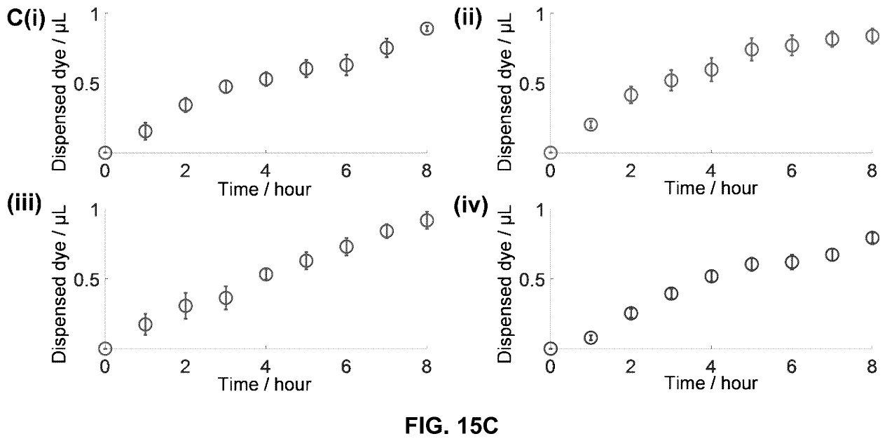

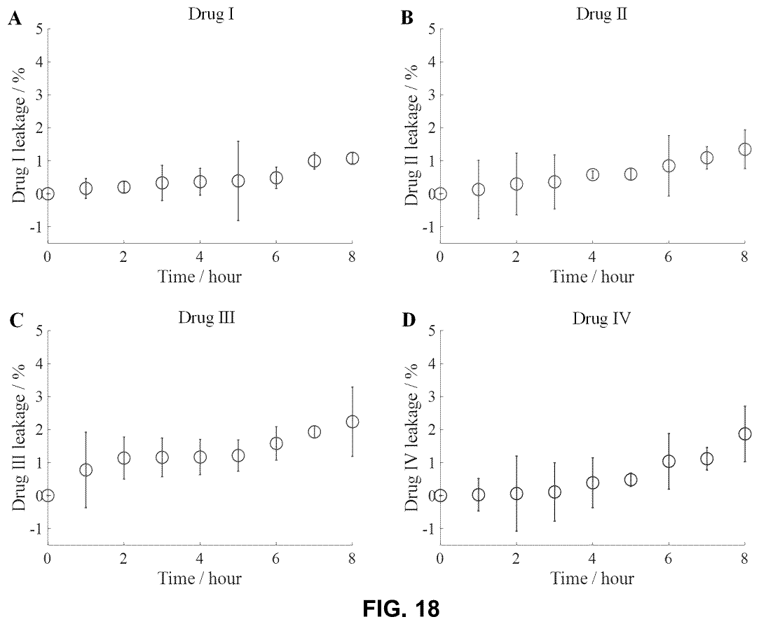

The drug-dispensing capabilities of the proposed soft robot were further evaluated by replacing the simulated drugs in the drug modules with fluorescent dyes (colloids of 200 nm fluorescent nanoparticles from Hai'an Zhichuan Battery Material Technology). The viscosity of this type of fluorescent dye was evaluated to be 13,200 μPa·s. To evaluate the robustness of the drug modules, the modules were controlled to dispense in a buffer solution (phosphate-buffered saline from XiGene Life Science, pH: 7.2-7.4). The viscosity of the buffer solution was evaluated to be 15,200 μPa·s. Two types of experiments were conducted based on such an experimental set up. For the first type of experiments, the objective was to investigate if the drug modules were still able to dispense their fluorescent dyes selectively. Hence, all the drug modules were actuated by four different sets of alternating \vec{B} (FIG. 15A). Although the buffer solution was more viscous than the simulated drug, the drug modules were still able to dispense successfully (FIG. 15A). Furthermore, similar to the outcome in FIG. 14A, each drug module would only dispense if and only if a designated, alternating \vec{B} of selected fand Bₐₘₚ was applied, indicating that the drug modules could dispense selectively (FIG. 15A). During the experiments, no observable leakage had been detected too. Because the view under the fluorescence microscope was unable to observe all the drug modules in this experiment, standard cameras were used to record these dispensing processes. The objective of the second type of experiments was to characterize the dispensing rates of all the drug modules when they dispense the fluorescent dye as the simulated drug in the buffer solution (see later section “Drug-dispensing capabilities”). Three sets of experiments were conducted for each drug module. In these experiments, the drug modules were commanded to dispense the drug over a period of eight hours. At an hourly interval, a fluorescence microscope was used to image the concentration of drugs in the modules (FIG. 15B). The light intensities at the regions of the fluorescent images, which overlap with the robot's structure in the module, were filtered off to obtain more accurate measurements for the drug-dispensing characterizations. From these experiments, the volume of drugs dispensed by all the modules across eight hours (FIG. 15C) were measured. A notable observation was that all of the drug modules were able to dispense 0.254-0.414 μL of drugs within 2 hours and such dispensing rates are generally practical for targeted drug delivery treatments. The mean dispensing rates for all the modules were 0.0992-0.115 μL/hour across 8 hours (FIG. 15C). It was noted that the drug-dispensing rates were lower than those in FIG. 14C. A plausible explanation of this reduction in dispensing rates was that the viscosity of the buffer solution in FIGS. 15B and 15C is 17 folds higher than those of the water reservoir in FIGS. 14B and 14C. Nonetheless, the experimental results in FIGS. 14A to 15C suggested that the drug-dispensing mechanism was robust because the drug modules were able to dispense successfully in different aquatic environments and with different drug contents. Furthermore, additional experiments were conducted to quantify the volume of leakage from the drug modules when they were not actuated in a buffer solution. In these experiments, all the drug modules were fully loaded with fluorescent dye and were placed in a buffer solution for eight hours. Across this eight-hour period, fluorescence microscopy was used to image the drug concentration within the modules at an hourly interval (FIG. 18). As three sets of experiments were performed for each drug module, each data point in FIG. 18 is evaluated with three trials, and the centroid and error bar of these data points represent their corresponding mean and standard deviations. The experimental results indicated that only 1.07-2.23% of the drugs were discharged after eight hours. This amount of leakage was negligible compared to the dispensing amount measured in the experiment shown in FIG. 15C.

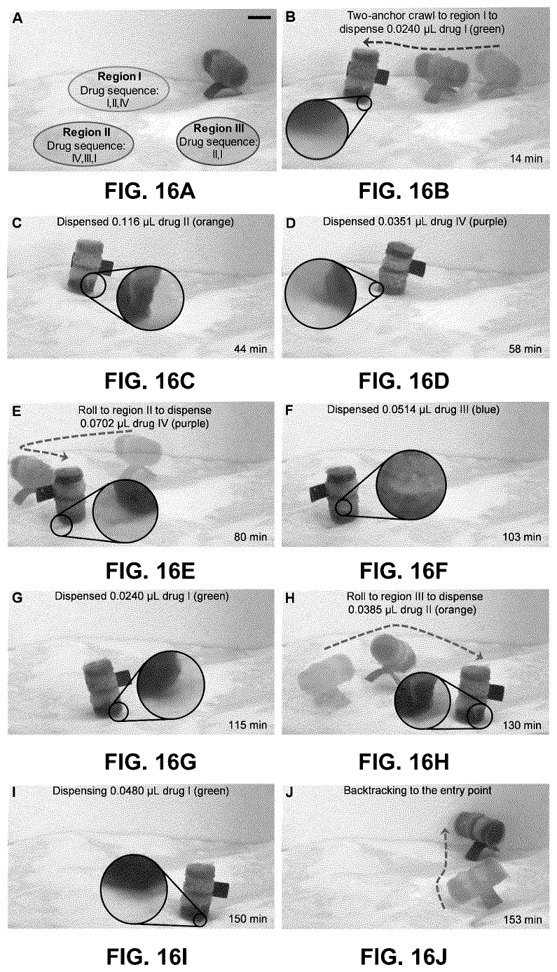

The potential of deploying the proposed soft robot for advanced targeted combination therapies is illustrated via the experiment in FIGS. 15A to 15C. For this experiment, the objective was to command the proposed soft robot to dispense specific sequence and dosage of drugs at three different regions of a biological platform (FIG. 16A). Each region would require the robot to deliver two to three distinct drugs and all the four types of drugs on the robot were required to be dispensed in this experiment (FIG. 16A). To have a better observation on the proposed soft robot's drug-dispensing processes, the robot was loaded with the colored drugs I-IV that are shown in FIG. 12A and not the fluorescent dyes. By using its rolling and two-anchor crawling gaits, the proposed soft robot could easily reach these targeted regions (FIG. 16A). In addition, since the proposed soft robot could reprogram its drug-dispensing dosage and sequence, it was able to successfully deliver the desired sequences and dosage of drugs at all the three regions (FIGS. 16B to 16I). As the color contrast between drug I and the phantom was low under certain lighting conditions, it may be challenging to observe drug I as it was dispensed in FIGS. 16B and 16I. However, it was observed that this drug had indeed been dispensed on the substrate. The proposed soft robot requires 153 minutes to complete the experiment in FIGS. 16A to 16I. In theory, such dispensing rates of the proposed soft robot were sufficient for medical purposes because combination therapies administered via the passive delivery method generally require between 90 minutes and 2 days to complete one treatment session. To exit the phantom, the proposed soft robot could also backtrack to its entry point (FIG. 16J). This demonstration therefore indicated that the soft robot could realize unprecedented reprogrammable drug-dispensing abilities since existing small-scale untethered actuators are unable to transport more than three types of drugs, and change their dispensing sequence and dosage. By enabling such reprogrammable abilities, miniature robots will now have great potential towards performing advanced targeted combination therapies via the active delivery method.

### Materials and Methods

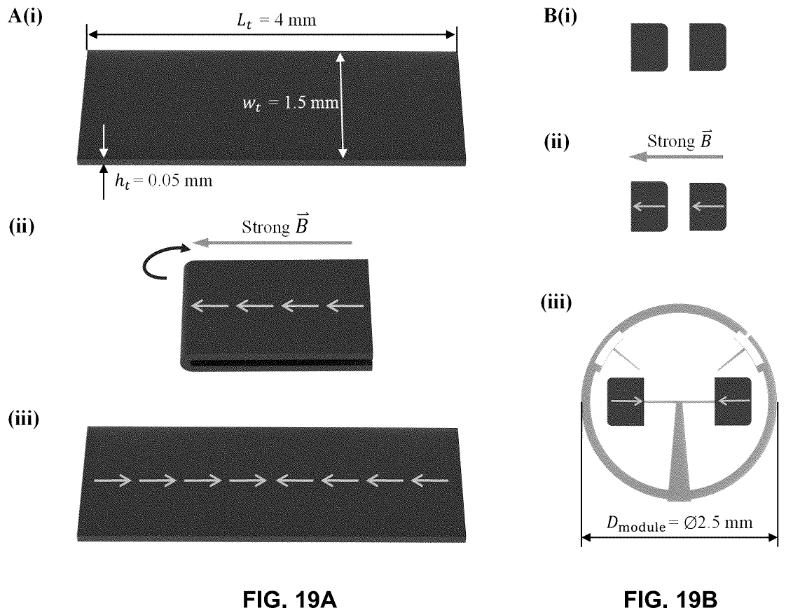

As shown in FIG. 12A, the proposed soft robot has four drug modules, an auxiliary magnet and a pair of soft tentacles. The soft tentacles in FIG. 19A(i) were made by embedding the PDMS polymer matrix (Dow Corning) with NdFeB magnetic microparticles (Magnequench: average size of 5 μm). The mass ratio between the NdFeB magnetic microparticles and PDMS is 3:1, while the elastomer base and curing agent of the PDMS had a mass ratio of 7:1. Each drug module has magnetized parts as well as passive components that did not respond to magnetic fields. For the magnetized parts in the drug modules and the auxiliary magnet of the robot, they were made by embedding NdFeB microparticles (Magnequench: average size of 5 μm) into a PDMS polymer matrix based on a 1:1 mass ratio (FIG. 19B(i)). The elastomer base and curing agent of this PDMS matrix had a corresponding mass ratio of 7:1. The passive components in the drug modules were made of pristine PDMS, which had an elastomer base to curing agent mass ratio of 10:1. Pristine PDMS is known to be biocompatible. The highest concentration of magnetic microparticles which were embedded into the PDMS matrix has the following NdFeB particles to PDMS mass ratio: 3:1, i.e., the smart magnetic composite that constructs the soft tentacles. This was due to the challenge faced when mixing the particles uniformly into the polymer matrix once the amount of particles exceeded this mass ratio. For the auxiliary magnet, it would be possible to further increase its magnetic microparticle loading.

All the components of the soft robot were molded separately, and were cured in an oven for two hours at 80° C. All of the molds were fabricated by photolithography methods. In general, photolithography methods can produce accurate molds with micrometer features, and such features are sufficient to create all of the components that are also in this range of size. Since it is possible to create reliable molds, the geometries of the components can be fabricated accurately. This allowed precise control over the stiffness and fundamental frequency of the vibrating beams and to endow the necessary stiffness properties for the tentacles. It is noteworthy that the geometry and Young's modulus of the vibrating beams are the key parameters that dictate the stiffness of the beams. Likewise, the stiffness and mass distribution of the vibrating beams will dictate their fundamental frequency. After all the components of the robot were cured, the magnetic components were first magnetized by a strong, uniform field of 1.1 T (FIGS. 19A(ii) and 19B(ii)). Subsequently, the components were assembled within the drug modules via adhesive means (FIG. 19B(iii)). The four drug modules were then adhesively bonded together with the tentacles to form the proposed soft robot (FIG. 12A). Once the soft robot was fully assembled, each drug module was loaded with its corresponding drug via injection. The drugs within the soft robot could also be reloaded upon demand. Because the stiffness and magnetic properties of the proposed soft robot plays a role in the theoretical model, experiments were conducted to measure these properties. For example, using a standard compression test (SHIMADZU AG-X plus, 10 kN), the Young's modulus of the magnetic soft beam and soft tentacles was evaluated to be 3.10±0.0877 MPa and 9.31±0.0193 MPa, respectively. These measurements were obtained from three trials.

Likewise, the magnetization magnitude (per volume) of the tentacles was measured by the vibrating sample magnetometer to be 84,100±386 A m⁻¹ with five trials. The magnetization magnitude (per volume) of the auxiliary magnet and the magnetized parts in the drug modules was measured by the vibrating sample magnetometer to be 37,600±1,802 A m⁻¹ too (with five trials). To evaluate the density of the magnetized and passive components in the drug modules, cuboid samples of 72 mm³ were created with these materials and their weight were measured with a precision scale (A&D GH-120). The density of these materials could then be computed via dividing their measured mass by the specified volume. The density of the magnetized and passive components of the vibrating beams in the drug modules were measured to be 3,060±168 kg m⁻³ (five trials) and 1,193±38 kg m⁻³ (five trials), respectively. These densities were used in the vibration analyses. Using standard rheology measurement tests (Bohlin gemini HR nano rheometer), the viscosity of the buffer solution and fluorescent dye were evaluated to be 15,200±1,150 μPa·s and 13,200±2,415 μPa·s, respectively. These measurements were obtained from three trials.

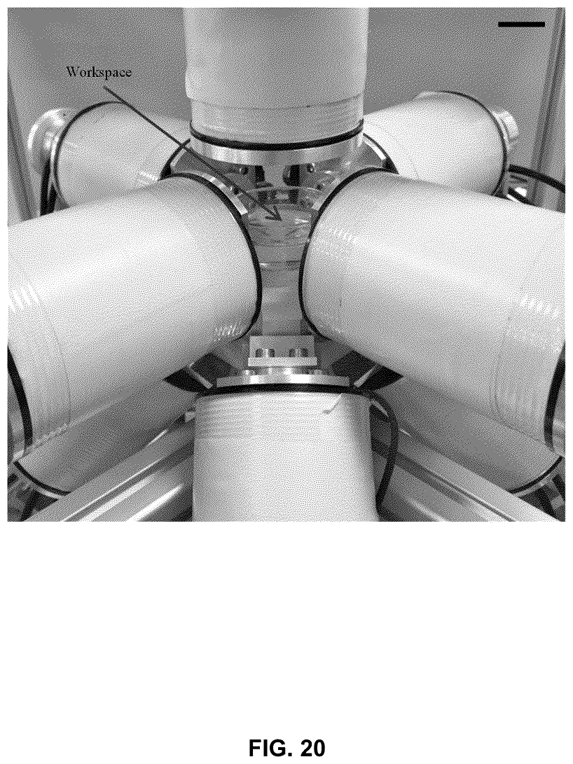

In all of the experiments, the liquid reservoir, which contains the proposed soft robot, was placed at the center of a customized electromagnetic coil system (FIG. 20). the electromagnetic coil system was able to generate a 90% homogeneous field in a workspace of 16 mm×16 mm×16 mm. Within this workspace, the coil system could output a maximum |\vec{B}| and magnetic spatial gradients of 30 mT and 0.4 T/m, respectively. Because the bandwidth of all the coils was higher than 210 Hz (Table 2), the coil system was able to produce the necessary alternating magnetic fields for commanding the proposed soft robot to dispense its drugs (2-19 mT and 20-180 Hz). The electromagnetic coils were at least 17 cm away from the proposed robot in all of the experiments.

TABLE 2
Electrical parameters for each coil of our
customized electromagnetic coil system.

| Resistance, R_{c} | Induction, L_{c} | Bandwidth |
|---|---|---|
| (Ω) | (mH) | (Hz) |
| 0.750-0.790 | 0.630-1.44 | 212-507 |

### Drug-Dispensing Capabilities

A. Dispensing Drugs from Selective Modules

For the experiments in FIGS. 14A to 14C, four additional drug modules were created that were identical to those of the proposed soft robot. These drug modules were placed on a flat substrate, and they are subjected to four different sets of alternating \vec{B}. In general, the alternating \vec{B} are periodic such that they are positive step functions for the first half of the period and their polarity would be reversed for the remaining period. The key differences between each set of alternating \vec{B} are that they have different frequencies and step amplitudes (Table 3). Each set of alternating \vec{B} is applied on the drug modules for three hours. Based on these experiments, it was observed that each set of alternating \vec{B} will only activate their selected drug module to dispense its contents. Therefore, this implies that it is viable to control the f and Bₐₘₚ of the alternating \vec{B} to command the soft robot to dispense a specific drug and such abilities would be essential for the soft robot to reprogram its drug-dispensing sequence and dosage.

TABLE 3
The frequency (f) and amplitude (Bₐₘₚ) of the alternating \vec{B}

| Drug module | Frequency f (Hz) | Amplitude Bₐₘₚ (mT) | $\begin{matrix}Maximum \frac{{dB}_{output}}{dt} \\ ( T {s}^{- 1} )\end{matrix}$ | $\begin{matrix}The threshold for the \\ maximum allowable \frac{d \vert \vec{B} \vert}{dt} thres \\ ( T {s}^{- 1} )\end{matrix}$ |
|---|---|---|---|---|
| I | 20 | 2 | 5.01 | 56.3 |
| II | 50 | 6 | 15.0 | |
| III | 100 | 12 | 30.1 | |
| IV | 180 | 19 | 47.6 | |

### B. Characterization of Drug-Dispensing Rates (Drugs I-IV on Flat Substrates)

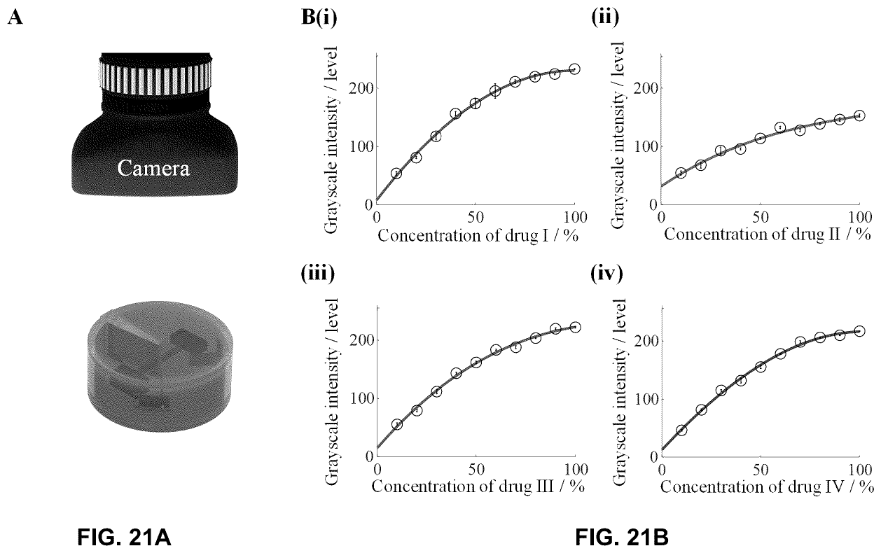

To measure the dispensing rate of each drug module, the modules were commanded to dispense their corresponding drug on a flat substrate over a period of eight hours. As the modules dispensed their drugs, computer vision techniques were used constantly to observe how the concentration of the drugs will change over time (FIG. 21A). To use such computer vision techniques, the relationship between the drugs' concentration and their corresponding grey level intensity was established (FIG. 21B) prior to conducting the experiments in FIG. 21A. The lighting conditions in our experiments are similar across the experiments so that the computer vision techniques can be executed reliably.

As shown in FIG. 14C, drug modules I-IV were able to dispense 46.9% (1.150 μl), 75.2% (1.849 μl), 50.2% (1.235 μl) and 68.8% (1.692 μl) of their respective drug after 8 hours. Each experiment in FIG. 14C was evaluated with three trials. To evaluate the practicality of our soft robot for advanced targeted combination therapy, we also measure the volume of drugs that it can dispense within 2 hours. The data in FIG. 14C indicated that the soft robot can dispense 0.288-0.462 μl of drugs within 2 hours, and such dispensing rates are generally sufficient for clinical treatments. For instance, the combination therapies for bladder cancer and ovarian carcinoma generally require approximately 0.0240-0.210 μl of drugs to be delivered to the disease site within 2 hours, and such requirements are within the limits of the proposed soft robot's dispensing rates.

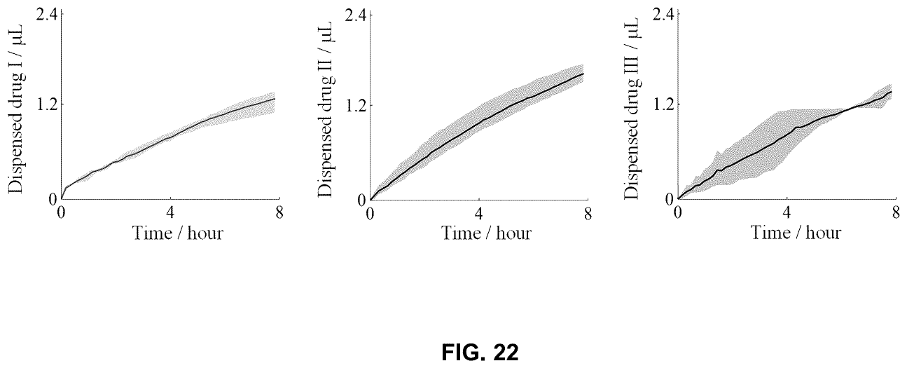

Each drug module of the proposed soft robot is not constrained to operate with a specific drug. Instead, the drug modules can still selectively dispense their drugs even when they are loaded with different drugs. For instance, after drugs I, II and III are loaded into drug module IV, these drugs will only be dispensed if and only if this drug module is actuated by its operating alternating \vec{B} (f: 180 Hz, Bₐₘₚ: 19 mT). Otherwise, the drugs will remain in the module. The dispensing rate of drug module IV, however, will change when it is loaded with different drugs. The dispensing rate of this drug module and the results are shown in FIG. 22. Each experiment in FIG. 22 was evaluated with three trials. In summary, drug module IV is able to dispense 52.8% of drug I (1.29 μl), 66.7% of drug II (1.64 μl) and 56.6% of drug III (1.39 μl) after 8 hours. The concentration of the drugs in these experiments was measured by our aforementioned computer vision techniques. The experiments in FIG. 22 suggested that the drug-dispensing mechanism of our soft robot is robust as its drug modules remain operational even after their drug contents have changed. These experimental results also suggested that it is necessary to characterize the dispensing rate of the modules when they are loaded with different drugs.

### C. Dispensing Drugs on Slopes (Drugs I-IV)

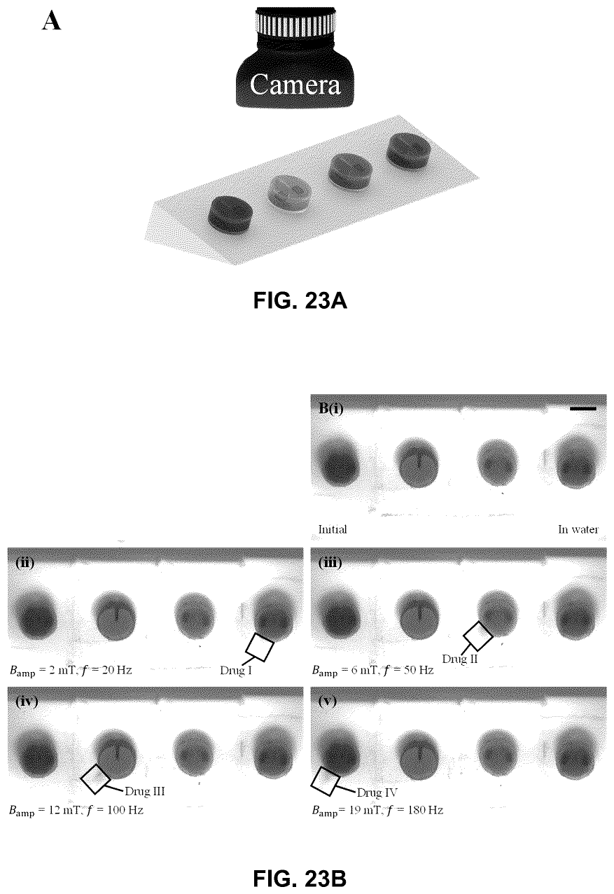

The drug modules of the soft robot can also selectively dispense their drugs on inclined slopes of differing grades (see FIGS. 23A and 23B). Specifically, the drug modules can accomplish such tasks on slopes that are between 5° and 20°. It is observed that when the specified alternating \vec{B} of drug module I (f:20 Hz, Bₐₘₚ:2 mT) was applied along the robot's \vec{m}, only drug module I will dispense its drugs. By applying the specified alternating \vec{B} of drug module II (f: 50 Hz, Bₐₘₚ: 6 mT), it was possible to selectively activate drug module II to dispense its drugs. Likewise, the alternating \vec{B} may be controlled such that our soft robot can dispense drugs from its modules III and IV on the slopes.

### D. Characterization of Drug-Dispensing Rates in a Buffer Solution (Fluorescent Dyes on Flat Substrates)

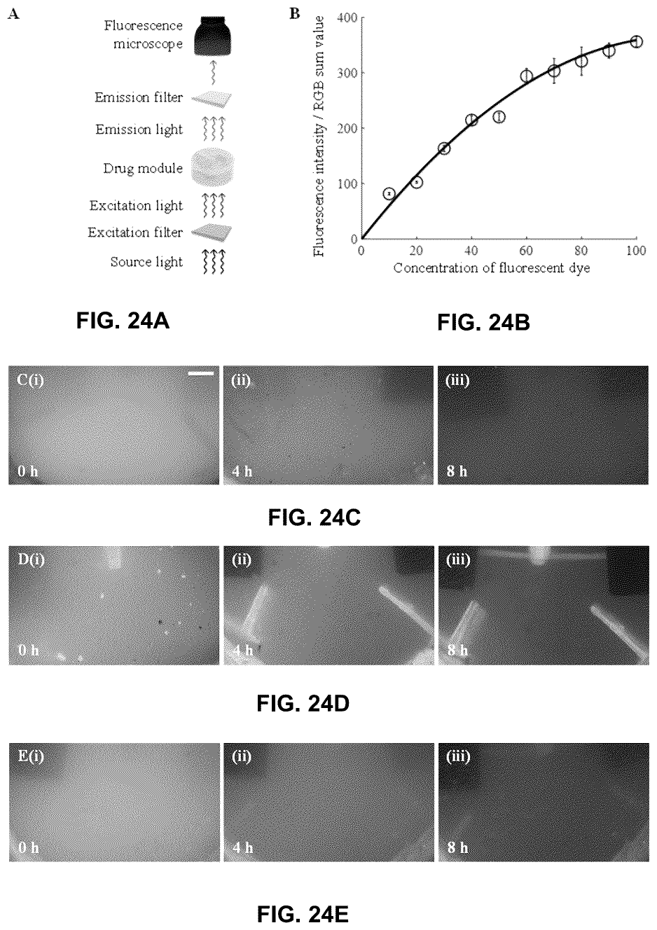

The drug-dispensing rates of the drug modules in a buffer solution (phosphate-buffered saline from XiGene Life Science, pH: 7.2 to 7.4, viscosity: 15,200 μPa·s) was evaluated when fluorescent dyes (colloids of 200 nm fluorescent nanoparticle from Hai'an Zhichuan Battery Material Technology) were used as the simulated drug. These dyes were able to absorb electromagnetic waves of 300-550 nm wavelength, and emit electromagnetic waves of 480-650 nm wavelength. By using a fluorescent microscope, electromagnetic waves of 515 nm wavelength emitted by the excited dyes may be detected via an emission filter after they are excited by electromagnetic waves of 465-495 nm wavelength (FIG. 24A). The intensity of the light observed under the fluorescent microscope has a positive correlation with the concentration of fluorescent dyes. Before conducting these experiments, the relationship between the fluorescent dyes' concentration and their corresponding light intensity were established (FIG. 24B). Three sets of experiments were then conducted for each drug module. In these experiments, the drug modules were commanded to dispense the drug over a period of eight hours. At an hourly interval, the fluorescence microscopy was used to image the drug concentration of the modules. The light intensities at the regions of the fluorescent images, which overlap with the robot's structure in the module, have been filtered off to obtain more accurate measurements for our drug-dispensing characterizations. From these experiments, we are able to measure the volume of drugs dispensed by all the modules across eight hours. As an example, FIG. 15B provides the fluorescent images of drug module I at the 0-, 4- and 8-hour intervals. The obtained drug-dispensing experimental results have been plotted in FIG. 15C. A notable observation is that all of our drug modules are able to dispense 0.254-0.414 μL of drugs within 2 hours and such dispensing rates are generally practical for targeted drug delivery treatments. The mean dispensing rates for all the modules were also measured to be 0.0992-0.115 μL/hour across 8 hours (FIG. 15C). It was noted that the drug-dispensing rates were lower than those in FIG. 14C (0.144-0.231 μL/hour across 8 hours). A plausible explanation of this reduction in dispensing rates is that the viscosity of the buffer solution in FIGS. 15B and 15C was 17 folds higher than those of the water reservoir in FIGS. 14B and 14C. Additional samples for the fluorescent images of our drug-dispensing experiments are shown in FIGS. 24C to 24E too.

### Exemplary Control Magnetic Field

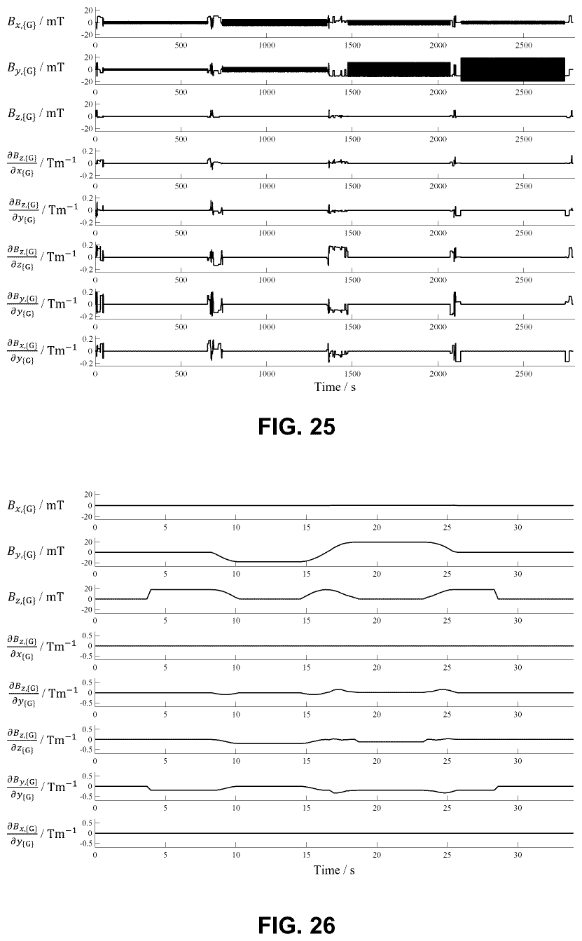

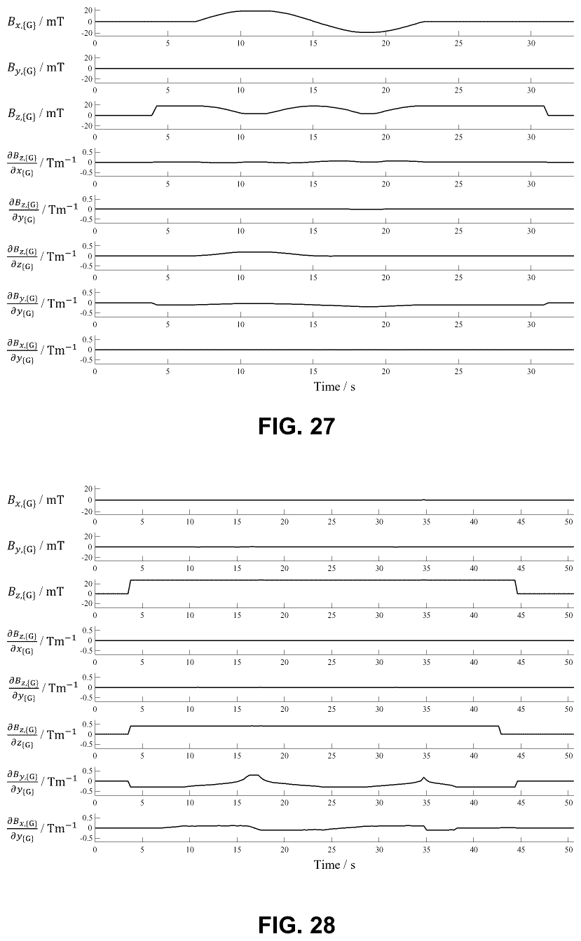

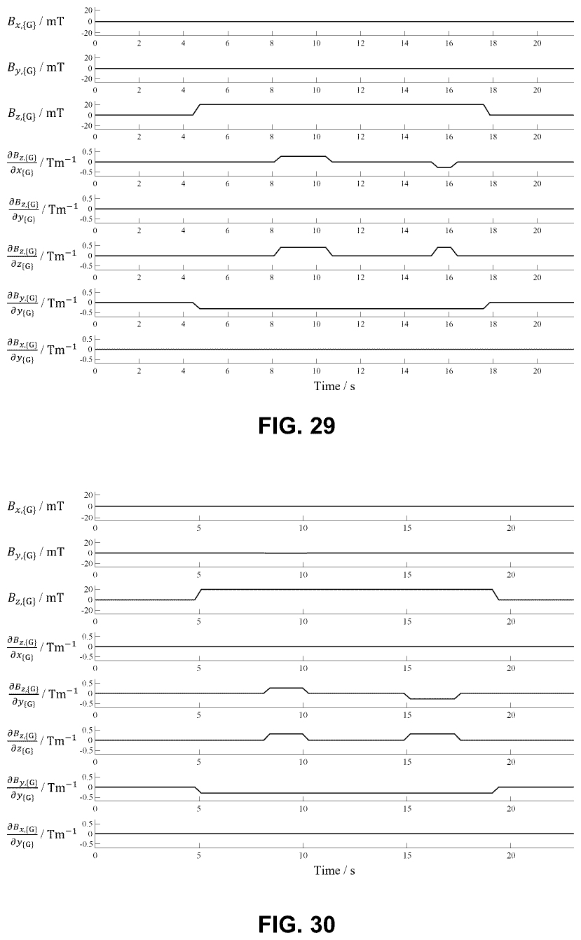

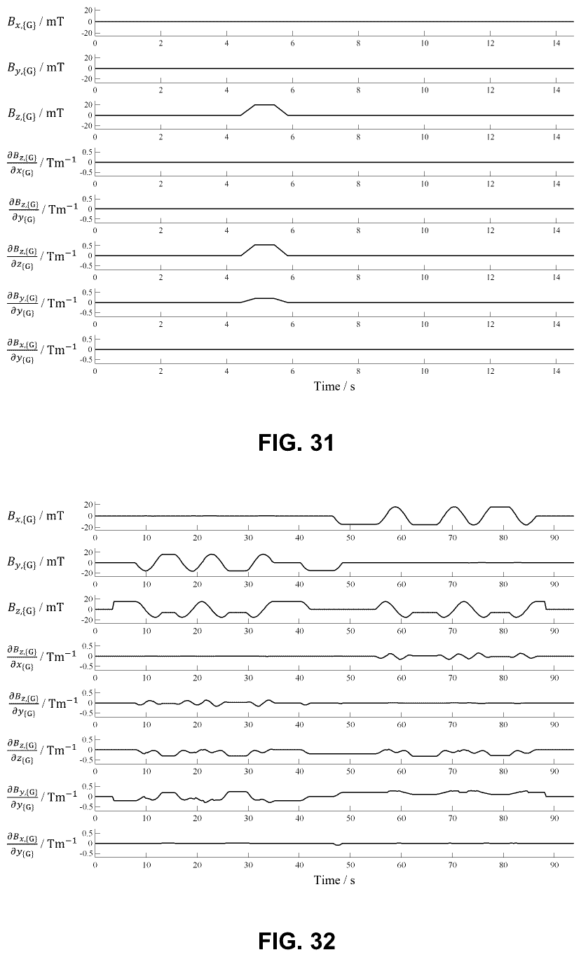

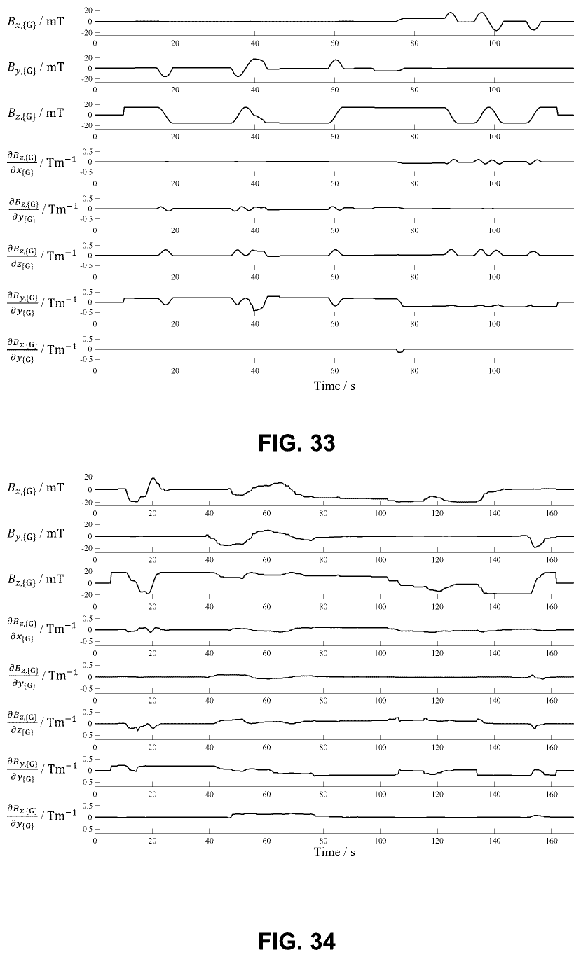

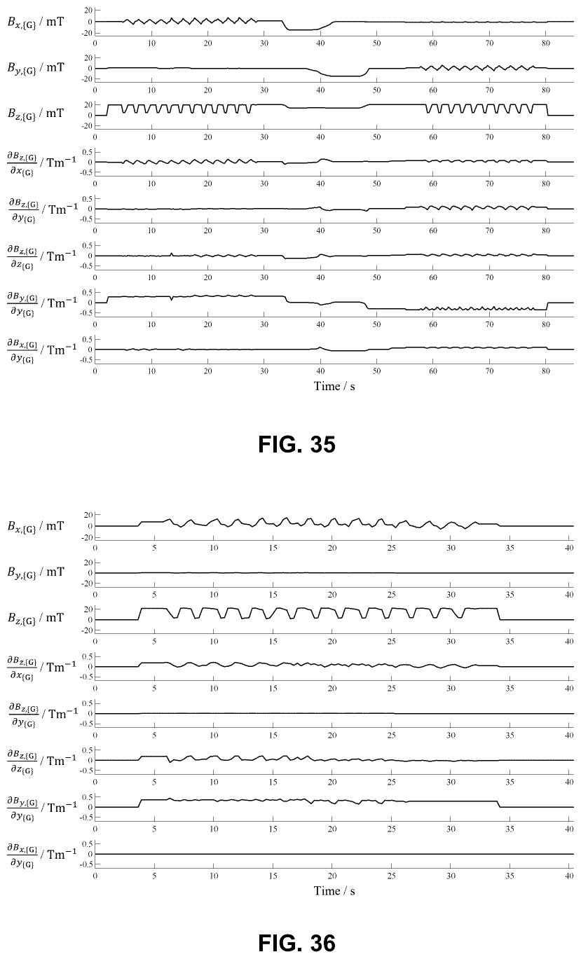

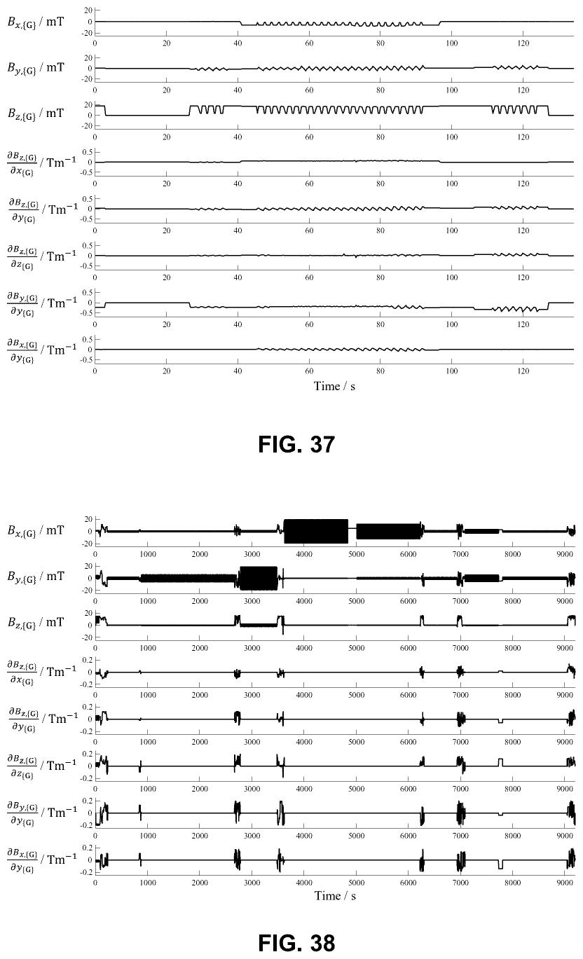

FIGS. 25 to 38 show various exemplary control magnetic field/control magnetic signals for the proposed robot. As examples, FIG. 25 shows an exemplary control magnetic signal for controlling the robot; FIG. 26 shows an exemplary control magnetic signal for x_{{L}}-axis rotation; FIG. 27 shows an exemplary control magnetic signal for y_{{L}}-axis rotation; FIG. 28 shows an exemplary control magnetic signal for z-axis rotation; FIG. 29 shows an exemplary control magnetic signal for x_{{L}}-axis translation; FIG. 30 shows an exemplary control magnetic signal for y_{{L}}-axis translation; FIG. 31 shows an exemplary control magnetic signal for z_{{L}}-axis translation; FIG. 32 shows an exemplary control magnetic signal for steering and rolling along the soft robot's length; FIG. 33 shows an exemplary control magnetic signal for steering and rolling along the soft robot's width; FIG. 34 shows an exemplary control magnetic signal for rolling across the barriers; FIG. 35 shows an exemplary control magnetic signal for steering the two-anchor crawling gait; FIG. 36 shows an exemplary control magnetic signal for using the two-anchor crawling gait to ascend a slope; FIG. 37 shows an exemplary control magnetic signal for using the two-anchor crawling gait to negotiate across the barrier; and FIG. 38 shows an exemplary control magnetic signal for maneuvering the robot.

### DISCUSSION

In general, it is much more favorable to have one miniature robot that can transport four distinct drugs with reprogrammable sequence and dosage compared to sequentially deploying four miniature robots where each of them can only carry a unique drug. This is because the latter type of treatments requires a relatively more invasive procedure since the robots must continuously access the human body. For this first study, a slightly larger prototype of the proposed soft robot was built for easy characterization purposes. Nevertheless, the current millimeter-scale prototype is theoretically sufficient for combination therapies in the gastrointestinal tracts since such treatments can be administered by centimeter-scale robots. Further miniaturization may be performed to reduce the proposed soft robot's length to 1.5 mm so that it can potentially access the central nervous system of up to 85% of the human population, and the ureter and urethra of adult humans. At the size of 1.5 mm, the proposed robot can theoretically carry sufficient drugs for targeted combination therapies to treat brain tumor, prostate cancer and colorectal cancer. It is also possible for the proposed robot to selectively dispense more than four types of drugs, but the additional drug modules will inevitably increase its overall size. The trade-off between the robot's size and its drug capacity will therefore be a critical considering factor for the future design iterations.

In summary, proposed herein a miniature soft robot formed from smart magnetic composites with unique stiffness and magnetization profiles. An unprecedented capability of the robot is that it can transport four distinct drugs, and its drug-dispensing sequence and dosage can be reprogrammed at multiple locations. Likewise, the proposed soft robot is considerably more dexterous than existing five-DOF miniature robots that have drug-dispensing capabilities because it has full six-DOF motions. The miniature soft robot demonstrates a significant advancement for smart magnetic composites and small-scale robotic technologies, and towards enabling advanced targeted combination therapies via active delivery methods.

All examples described herein, whether of methods, materials, or products, are presented for the purpose of illustration and to aid understanding and are not intended to be limiting or exhaustive. Modifications may be made by one of ordinary skill in the art without departing from the scope of the invention as claimed.

1. A liquid dispensing robot, comprising:
a plurality of dispensing modules, each of the plurality of dispensing modules comprising:
a housing defining an inner chamber and an opening, the opening allowing fluid communication between the inner chamber and an environment;
an actuating member moveably coupled to the housing, the actuating member disposed in the inner chamber defining an actuation frequency band; and
a dispensing member movably coupled to the housing, the dispensing member disposed in the inner chamber adjacent to the opening,
wherein responsive to a control magnetic field with a dispensing frequency within the actuation frequency band, the actuating member deforms to periodically displace the dispensing member relative to the opening to vary a local fluid pressure adjacent to the opening,
wherein each of the actuation frequency band is non-overlapping with one another,
wherein each of the actuating member of the plurality of dispensing modules is selectively actuatable by the control magnetic field.

2. The liquid dispensing robot as recited in claim 1, wherein the dispensing member is spaced apart from the opening.

3. The liquid dispensing robot as recited in claim 1, wherein for each of the plurality of dispensing modules, the actuating member is spaced apart from the dispensing member in the inner chamber when the dispensing frequency is outside the actuation frequency band.

4. The liquid dispensing robot as recited in claim 1, wherein for each of the plurality of dispensing modules, the actuating member comprises a pair of beams and at least one magnetized mass coupled to each of the pair of beams.

5. The liquid dispensing robot as recited in claim 1, wherein for each of the plurality of dispensing modules, the actuating member comprises at least a coupling portion fixedly coupled to the housing.

6. The liquid dispensing robot as recited in claim 1, wherein for each of the plurality of dispensing modules, periodically displacing the dispensing member relative to the opening increases the local fluid pressure of a first liquid adjacent to the opening.

7. The liquid dispensing robot as recited in claim 6, wherein the local fluid pressure exceeding a threshold pressure, causes the first liquid in the inner chamber to exit through the opening from the inner chamber to the environment along a liquid flow axis.

8. The liquid dispensing robot as recited in claim 7, wherein periodically displacing the dispensing member causes a second liquid in the environment to enter through the opening from the environment to the inner chamber along the liquid flow axis.

9. The liquid dispensing robot as recited in claim 7, wherein for each of the plurality of dispensing modules, the dispensing member is at least partially displaceable along the liquid flow axis.

10. The liquid dispensing robot as recited in claim 6, wherein for each of the plurality of dispensing modules, the opening is sized to provide a fluid resistance such that the first liquid is contained within the inner chamber in the absence of a displacement of the dispensing member.

11. The liquid dispensing robot as recited in claim 1, further comprising a locomotion module, the locomotion module being configured to move the liquid dispensing robot responsive to the control magnetic field with a locomotion frequency, the locomotion frequency non-overlapping with each of the actuation frequency band.

12. The liquid dispensing robot as recited in claim 11, wherein the locomotion module defines a locomotion magnetic dipole in line with a magnetic axis of the robot, wherein the actuating member and the locomotion module collectively defines a net magnetic moment aligned with the magnetic axis.

13. The liquid dispensing robot as recited in claim 11, wherein the actuating member is biased away from the dispensing member responsive to the control magnetic field with the locomotion frequency, wherein the locomotion frequency corresponds to a quasistatic magnetic field.

14. The liquid dispensing robot as recited in claim 11, wherein the magnetized locomotion module comprises a pair of tentacles symmetrically arranged about the magnetic axis.

15. The liquid dispensing robot as recited in claim 11, wherein the plurality of dispensing modules are disposed adjacent to one another along a longitudinal axis, the longitudinal axis orthogonal to the magnetic axis.

16. A liquid dispensing robotic system, comprising: an electromagnetic coil system defining a workspace; and the liquid dispensing robot as recited in claim 1 disposed in the workspace, the electromagnetic coil system being configured to provide the control magnetic field.

17. A method of liquid dispensing, the method comprising:
providing a control magnetic field with a first dispensing frequency to the liquid dispensing robot as recited in claim 1; and
providing the control magnetic field with a second dispensing frequency to the liquid dispensing robot,
wherein the first dispensing frequency is within a first actuation frequency band and the second dispensing frequency is within a second actuation frequency band,
wherein the first actuation frequency band is non-overlapping with the second actuation frequency band.

18. The method of liquid dispensing as recited in claim 17, wherein the first dispensing frequency and the second dispensing frequency of the control magnetic field are independently provided non-overlapping in time.

19. The method of liquid dispensing as recited in claim 17, further comprising: providing the control magnetic field with a locomotion frequency to move the liquid dispensing robot to a target position, the locomotion frequency non-overlapping with each of the first actuation frequency band and the second actuation frequency band.

20. The method of liquid dispensing as recited in claim 19, wherein the control magnetic field comprises at least one of: the first dispensing frequency and the second dispensing frequency provided at least partially concurrent with the locomotion frequency.
